(19) United States
(12) Patent Application Publication
(10) Pub. No.: US 2024/0114772 A1
(43) Pub. Date: Apr. 4, 2024

(54) ORGANIC ELECTROLUMINESCENT DEVICE

(71) Applicant: SFC CO., LTD., Cheongju-si, Chungcheongbuk-do (KR)

(72) Inventors: Soon Wook Cha, Cheongju-si (KR); Ji Won Lee, Cheongju-si (KR)

(21) Appl. No.: 18/274,948

(22) PCT Filed: Jan. 28, 2022
(86) PCT No.: PCT/KR2022/001659

§ 371 Date: Jul. 28, 2023

(30) Foreign Application Priority Data
Jan. 29, 2021 (KR) 10-2021-0013030

(51) Int. Cl. H10K 85/60; C07C 15/30; C09K 11/06

(52) U.S. Cl. CPC H10K 85/626; C07C 15/30; C09K 11/06; H10K 50/11

(57) ABSTRACT

An organic electroluminescent device according to the present invention is an organic electroluminescent device comprising a first electrode, a second electrode, and an organic material layer formed between the first electrode and the second electrode, wherein the organic material layer is formed using a composition having an organic electroluminescence material and a solvent, the organic electroluminescence material has a host, and the organic electroluminescence device has excellent operation properties such as luminous efficiency and driving voltage by using an organic electroluminescence material having high solubility in a solvent.

### TECHNICAL FIELD

The present invention relates to an organic electroluminescent device, and more particularly, to an organic electroluminescent device comprising an organic electroluminescent material having high solubility in a solvent, with improved operating properties of the device, such as luminous efficiency and driving voltage.

### BACKGROUND ART

An organic electroluminescent device, which is a display device using a self-emission phenomenon, has advantages such as a large viewing angle, lightness and simplicity compared to a liquid crystal display, and a fast response speed, and thus is expected to be used as a full-color display or lighting.

In general, the organic light emitting phenomenon refers to a phenomenon in which electric energy is converted into light energy by using an organic material. An organic electroluminescent device using the organic light emitting phenomenon generally has a structure including an anode, a cathode, and an organic material layer therebetween. Here, the organic material layer often consists of a multi-layer structure composed of different materials in order to increase the luminous efficiency and stability of the organic electroluminescent device, and for example, it may consist of a hole injecting layer, a hole transport layer, a light emitting layer, an electron transport layer, an electron injecting layer, etc. In the structure of the organic electroluminescent device, when a voltage is applied between the two electrodes, holes are injected into the organic material layer in the cathode and electrons are injected into the organic material layer in the anode, excitons are formed when the injected holes and electrons meet, and light is emitted when the excitons fall to a bottom state again. It is known that the organic light emitting device has properties such as self-emission, high luminance, high emission efficiency, low driving voltage, wide viewing angle, high contrast, high speed response, and the like.

Currently, displays are becoming larger and larger. When a large-sized display is manufactured using a deposition process, there are disadvantages such as a decrease in production yield and an increase in investment cost due to an increase in the size of a substrate. In addition, since the deposition process evaporates the monomolecular material under vacuum conditions and deposits the material on the substrate, there is a limitation that a material having a high glass transition temperature should be used so that decomposition does not occur at a high evaporation temperature.

On the other hand, when a large-sized display is manufactured by dissolving an organic electroluminescent material in a solvent to prepare a solution and then applying the solution on a base material, there are advantages that the process cost is lower and the process steps are relatively simple compared to the deposition process. However, since the organic electroluminescent material often has low solubility with respect to the solvent, it is difficult to secure light emitting efficiency, luminance, power efficiency, thermal stability, and lifespan characteristics of the device.

Therefore, there is a continuous need to develop an organic electroluminescent material having high solubility in a solvent and capable of improving light emitting efficiency, luminance, power efficiency, thermal stability, and lifespan properties of a device.

### DETAILED DESCRIPTION OF THE INVENTION

### Technical Task

An object of the present invention is to provide an organic electroluminescent device having a lower driving voltage and improved luminous efficiency by using an organic electroluminescent material having high solubility in a solvent.

The tasks to be achieved by the present invention are not limited to the above-mentioned task, and other tasks not mentioned will be clearly understood by those skilled in the art from the following description.

### Means for Achieving the Task

An organic electroluminescence device according to one embodiment of the present invention is an organic electroluminescence device comprising a first electrode, a second electrode, and an organic material layer formed between the first electrode and the second electrode, wherein the organic material layer is formed using a composition having an organic electroluminescence material and a solvent, the organic electroluminescence material has a host, and the host may be one or more compounds represented by the following Formula 1.

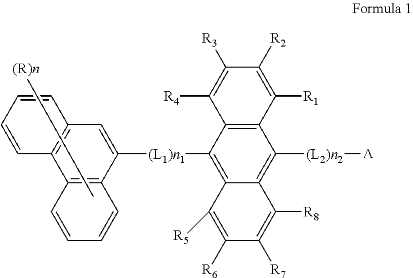

wherein in Formula 1, A is selected from the group consisting of a substituted or unsubstituted alkyl group having 1 to 30 carbon atoms, a substituted or unsubstituted alkenyl group having 2 to 30 carbon atoms, a substituted or unsubstituted cycloalkyl group having 3 to 30 carbon atoms, a substituted or unsubstituted cycloalkenyl group having 5 to 30 carbon atoms, a substituted or unsubstituted aryl group having 6 to 50 carbon atoms, and a substituted or unsubstituted heteroaryl group having 3 to 50 carbon atoms,
L1 and L2 are the same as or different from each other, and are each independently selected from the group consisting of a single bond, a substituted or unsubstituted arylene group having 6 to 50 carbon atoms, and a substituted or unsubstituted heteroarylene group having 3 to 50 carbon atoms,
n1 and n2 are respectively 1 or 2,
Rs are the same as or different from each other, and are each independently selected from the group consisting of a hydrogen atom, a deuterium atom, a substituted or unsubstituted alkyl group having 1 to 30 carbon atoms, a substituted or unsubstituted alkenyl group having 2 to 30 carbon atoms, a substituted or unsubstituted cycloalkyl group having 3 to 30 carbon atoms, a substituted or unsubstituted cycloalkenyl group having 5 to 30 carbon atoms, a substituted or unsubstituted alkoxy group having 1 to 30 carbon atoms, a substituted or unsubstituted aryloxy group having 6 to 30 carbon atoms, a substituted or unsubstituted alkylthioxy group having 1 to 30 carbon atoms, a substituted or unsubstituted arylthioxy group having 6 to 30 carbon atoms, a substituted or unsubstituted alkylamine group having 1 to 30 carbon atoms, a substituted or unsubstituted arylamine group having 6 to 30 carbon atoms, a substituted or unsubstituted aryl group having 6 to 50 carbon atoms, a substituted or unsubstituted heteroaryl group having 3 to 50 carbon atoms, a substituted or unsubstituted alkylsilyl group having 1 to 30 carbon atoms, and a substituted or unsubstituted arylsilyl group having 6 to 30 carbon atoms,
n is 9, and
R1 to R8 are the same as or different from each other, and are each independently selected from the group consisting of a hydrogen atom, a deuterium atom, a substituted or unsubstituted alkyl group having 1 to 30 carbon atoms, a substituted or unsubstituted alkenyl group having 2 to 30 carbon atoms, a substituted or unsubstituted cycloalkyl group having 3 to 30 carbon atoms, a substituted or unsubstituted cycloalkenyl group having 5 to 30 carbon atoms, a substituted or unsubstituted aryl group having 6 to 50 carbon atoms, a substituted or unsubstituted heteroaryl group having 3 to 50 carbon atoms, a substituted or unsubstituted arylamine group having 6 to 30 carbon atoms, a substituted or unsubstituted alkylsilyl group having 1 to 30 carbon atoms, and a substituted or unsubstituted arylsilyl group having 6 to 30.

### Effect of the Invention

The organic electroluminescence device according to the present invention may be manufactured using an organic electroluminescence material having high solubility in a solvent, and has excellent operation properties such as luminous efficiency and driving voltage.

### BRIEF DESCRIPTION OF THE DRAWINGS

FIG. 1 is a schematic cross-sectional view showing an organic electroluminescence device according to an embodiment of the present invention.

### DETAILED DESCRIPTION FOR CARRYING OUT THE INVENTION

Hereinafter, an organic electroluminescence device according to the present invention will be described with reference to preferred embodiments. However, these examples are intended to describe the present invention in more detail, and it will be apparent to those skilled in the art that the scope of the present invention is not limited thereto.

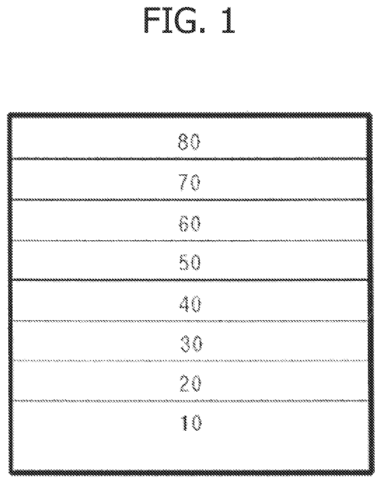

FIG. 1 is a schematic cross-sectional view showing an organic electroluminescence device according to an embodiment of the present invention.

Referring to FIG. 1, the organic electroluminescence device according to an embodiment of the present invention may comprise a first electrode 20, a second electrode 80 facing the first electrode 20, and an organic material layer formed between the first electrode 20 and the second electrode 80.

For example, the first electrode 20 may be an anode, and the second electrode 80 may be a cathode. The organic material layer may comprise at least one of a hole transport layer 40, an organic light emitting layer 50, and an electron transport layer 60. The organic material layer may further comprise a hole injecting layer 30 and an electron injecting layer 70 if necessary, and may further comprise single or multiple intermediate layers, other than the above.

The organic material layer may be formed by a deposition process or a solution process.

The deposition process may refer to, for example, a method of forming a thin film by evaporating a material substance in a vacuum or low-pressure state by a method such as heating. The solution process may refer to, for example, a method of forming a solution by mixing a material substance with a solvent, and then forming a thin film using the solution by a method such as spin coating, dip coating, doctor blade coating, spray coating, roll coating, inkjet printing, and screen printing.

A hole injecting layer (HIL) 30 may be provided between the first electrode 20 and the hole transport layer 40. The hole injecting layer 30 may include, for example, poly(3,4-ethylenedioxythiophene):poly(styrenesulfonate)(PEDOT:PSS), [4,4′,4″-tris(2-naphthylphenyl-phenylamino)-triphenylamine](2-TNATA), [N,N′-di(l-naphthyl)-N,N′-diphenylbenzidine)](NPD), [N,N′-diphenyl-N,N′-bis(3-methylphenyl)-1,1′-biphenyl-4,4′-diamine](TPD), [N,N′-diphenyl-N,N′-bis-[4-(phenyl-m-tolyl-amino)-phenyl]-biphenyl-4,4′-diamine](DNTPD), copper phthalocyanine, or a starburst type amine [4,4′,4″-tri(N-carbazolyl)triphenyl-amine](TCTA), [4,4′,4″-tris-(3-methylphenylamino)triphenylamine] (m-MTDATA), etc., but is not limited to thereto, and may include a material commonly used as a hole injecting layer in the pertinent art.

A hole transport layer (HTL) 40 may be provided between the hole injecting layer 30 and the organic light emitting layer 50. The hole transport layer 40 may comprise an electron-donating material having a small ionization potential. More specifically, the hole transport layer 40 may include a diamine, a triamine or a tetraamine derivative mainly having triphenylamine as a basic skeleton, for example, N,N′-bis(3-methylphenyl)-N,N′-diphenyl-[1,1-biphenyl]-4,4′-diamine (TPD) or N,N′-di(naphthalen-1-yl)-N,N′-diphenylbenzidine (a-NPD), etc.

An electron transport layer (ETL) 60 may be provided between the second electrode 80 and the organic light emitting layer 50. The electron transport layer 60 may include, for example, PBD, BMD, BND, or Alq3, etc. which are oxadiazole derivatives.

An electron injecting layer (EIL) 70 may be provided between the second electrode 80 and the electron transport layer 60. The electron injecting layer 70 may include, for example, LiF, NaCl, CsF, Li2O, BaO, or the like, but is not limited thereto, and may include a material generally used as an electron transport layer in the pertinent art.

The organic light emitting layer 50 may be provided between the hole transport layer 40 and the electron transport layer 60. The organic emission layer 50 may be formed using a composition containing an organic electroluminescent material and a solvent. The organic electroluminescent material may has a host, and the host may be at least one compound represented by Formula 1.

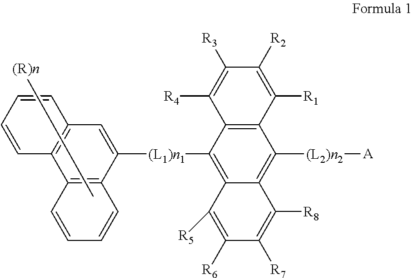

wherein in Formula 1, A is selected from the group consisting of a substituted or unsubstituted alkyl group having 1 to 30 carbon atoms, a substituted or unsubstituted alkenyl group having 2 to 30 carbon atoms, a substituted or unsubstituted cycloalkyl group having 3 to 30 carbon atoms, a substituted or unsubstituted cycloalkenyl group having 5 to 30 carbon atoms, a substituted or unsubstituted aryl group having 6 to 50 carbon atoms, and a substituted or unsubstituted heteroaryl group having 3 to 50 carbon atoms,
L1 and L2 are the same as or different from each other, and are each independently selected from the group consisting of a single bond, a substituted or unsubstituted arylene group having 6 to 50 carbon atoms, and a substituted or unsubstituted heteroarylene group having 3 to 50 carbon atoms,
n1 and n2 are respectively 1 or 2,
Rs are the same as or different from each other, and are each independently selected from the group consisting of a hydrogen atom, a deuterium atom, a substituted or unsubstituted alkyl group having 1 to 30 carbon atoms, a substituted or unsubstituted alkenyl group having 2 to 30 carbon atoms, a substituted or unsubstituted cycloalkyl group having 3 to 30 carbon atoms, a substituted or unsubstituted cycloalkenyl group having 5 to 30 carbon atoms, a substituted or unsubstituted alkoxy group having 1 to 30 carbon atoms, a substituted or unsubstituted aryloxy group having 6 to 30 carbon atoms, a substituted or unsubstituted alkylthioxy group having 1 to 30 carbon atoms, a substituted or unsubstituted arylthioxy group having 6 to 30 carbon atoms, a substituted or unsubstituted alkylamine group having 1 to 30 carbon atoms, a substituted or unsubstituted arylamine group having 6 to 30 carbon atoms, a substituted or unsubstituted aryl group having 6 to 50 carbon atoms, a substituted or unsubstituted heteroaryl group having 3 to 50 carbon atoms, a substituted or unsubstituted alkylsilyl group having 1 to 30 carbon atoms, and a substituted or unsubstituted arylsilyl group having 6 to 30 carbon atoms,
n is 9, and
R1 to R8 are the same as or different from each other, and are each independently selected from the group consisting of a hydrogen atom, a deuterium atom, a substituted or unsubstituted alkyl group having 1 to 30 carbon atoms, a substituted or unsubstituted alkenyl group having 2 to 30 carbon atoms, a substituted or unsubstituted cycloalkyl group having 3 to 30 carbon atoms, a substituted or unsubstituted cycloalkenyl group having 5 to 30 carbon atoms, a substituted or unsubstituted aryl group having 6 to 50 carbon atoms, a substituted or unsubstituted heteroaryl group having 3 to 50 carbon atoms, a substituted or unsubstituted arylamine group having 6 to 30 carbon atoms, a substituted or unsubstituted alkylsilyl group having 1 to 30 carbon atoms, and a substituted or unsubstituted arylsilyl group having 6 to 30, and
wherein in Formula 1, “substituted” in “substituted or unsubstituted” means substituted with one or more substituents selected from the group consisting of a deuterium, a cyano group, a halogen group, a hydroxyl group, a nitro group, an alkyl group having 1 to 24 carbon atoms, an alkenyl group having 1 to 24 carbon atoms, a cycloalkyl group having 3 to 24 carbon atoms, a heteroalkyl group having 1 to 24 carbon atoms, an aryl group having 6 to 24 carbon atoms, an arylalkyl group having 7 to 24 carbon atoms, an alkylaryl group having 7 to 24 carbon atoms, a heteroaryl group having 2 to 24 carbon atoms, a heteroarylalkyl group having 2 to 24 carbon atoms, an alkoxy group having 1 to 24 carbon atoms, an alkylamino group having 1 to 24 carbon atoms, a diarylamino group having 12 to 24 carbon atoms, a diheteroarylamino group having 2 to 24 carbon atoms, an aryl (heteroaryl) amino group having 7 to 24 carbon atoms, an alkylsilyl group having 1 to 24 carbon atoms, an arylsilyl group having 6 to 24 carbon atoms, an aryloxy group having 6 to 24 carbon atoms, and an arylthionyl group having 6 to 24 carbon atoms.

In addition, when considering the ranges of the alkyl group or the aryl group in ‘substituted or unsubstituted alkyl group having 1 to 24 carbon atoms’, ‘substituted or unsubstituted aryl group having 6 to 24 carbon atoms’, etc., the ranges of the number of carbon atoms of the alkyl group having 1 to 24 carbon atoms and the aryl group having 6 to 24 carbon atoms each mean the total number of carbon atoms constituting the alkyl portion or the aryl portion when viewed as being unsubstituted without considering the substituted portion of the substituent. For example, the phenyl group in which a butyl group is substituted at the para position should be regarded as corresponding to an aryl group having 6 carbon atoms substituted with a butyl group having 4 carbon atoms.

The aryl group of the compound of the present invention represented by Formula 1 means an aromatic system consisting of hydrocarbons including one or more rings, and when the aryl group has a substituent, the aryl group may be fused with neighboring substituents to further form a ring.

Specific examples of the aryl group may include aromatic groups such as phenyl group, o-biphenyl group, m-biphenyl group, p-biphenyl group, o-terphenyl group, m-terphenyl group, p-terphenyl group, naphthyl group, anthryl group, phenanthryl group, pyrenyl group, indenyl group, fluorenyl group, tetrahydronaphthyl group, perylenyl group, chrysenyl group, naphthacenyl group, fluoranthenyl group, triphenylenyl group, and the like.

The heteroaryl group which is the substitutent used in the compound of the present invention represented by Formula 1 means a C2-C24 ring aromatic system comprising 1, 2 or 3 heteroatoms selected from N, O, P, Si, S, Ge, Se or Te, and the remaining ring atoms being carbon, and the rings may be fused to form a ring. In addition, one or more hydrogen atoms in the heteroaryl group may be substituted with the same substituent as in the aryl group.

The alkyl group of the compound of the present invention represented by Formula 1 is linear or branched, and specific examples thereof include methyl, ethyl, propyl, isopropyl, isobutyl, sec-butyl, tert-butyl, pentyl, iso-amyl, hexyl, etc., and one or more hydrogen atoms in the alkyl group may be substituted with the same substituent as in the aryl group.

The heteroalkyl group of the compound of the present invention represented by Formula 1 means that one or more, preferably 1 to 5, carbon atoms in the main chain of the alkyl group are substituted with a heteroatom such as an oxygen atom, a sulfur atom, a nitrogen atom, a phosphorous atom, etc., and one or more hydrogen atoms in the heteroalkyl group may be substituted with the same substituent as in the alkyl group.

The ‘cyclo’ in the cycloalkyl group of the compound of the present invention represented by Formula 1 means a substituent having a structure capable of forming a single ring or multiple rings of saturated hydrocarbon in an alkyl group, and specific examples of the cycloalkyl group include cyclopropyl, cyclobutyl, cyclopentyl, cyclohexyl, methylcyclopentyl, methylcyclohexyl, ethylcyclopentyl, ethylcyclohexyl, adamantyl, dicyclopentadienyl, decahydronaphthyl, norbornyl, bornyl, isobornyl, etc., and one or more hydrogen atoms in the cycloalkyl group may be substituted with the same substituent as in the aryl group.

The alkoxy group of the compound of the present invention represented by Formula 1 is a substituent in which an oxygen atom is bonded to the terminal of an alkyl group or a cycloalkyl group, and specific examples thereof include methoxy, ethoxy, propoxy, isobutyloxy, sec-butyloxy, pentyloxy, iso-amyloxy, hexyloxy, etc., and one or more hydrogen atoms in the alkoxy group may be substituted with the same substituent as in the aryl group.

Specific examples of the arylalkyl group of the compound of the present invention represented by Formula 1 include phenylmethyl (benzyl), phenylethyl, phenylpropyl, naphthylmethyl, naphthylethyl, etc., and one or more hydrogen atoms in the arylalkyl group may be substituted with the same substituent as in the aryl group.

Specific examples of the alkylsilyl group of the compound of the present invention represented by Formula 1 include trimethylsilyl, triethylsilyl, methylcyclobutylsilyl, etc. and one or more hydrogen atoms in the alkylsilyl group may be substituted with the same substituent as in the aryl group.

Specific examples of the arylsilyl group of the compound of the present invention represented by Formula 1 include triphenylsilyl, diphenylmethylsilyl, diphenylvinylsilyl, etc., and one or more hydrogen atoms in the arylsilyl group may be substituted with the same substituent as in the aryl group.

The alkenyl group of the compound of the present invention represented by Formula 1 means an alkyl substituent comprising one carbon-carbon double bond consisting of two carbon atoms, and the alkynyl group means an alkyl substituent comprising one carbon-carbon triple bond consisting of two carbon atoms.

The diarylamino group of the compound of the present invention represented by Formula 1 means an amine group in which two identical or different aryl groups described above are bonded to a nitrogen atom, the diheteroarylamino group of the compound of the present invention means an amine group in which two identical or different heteroaryl groups are bonded to a nitrogen atom, and the aryl(heteroaryl)amino group means an amine group in which each of the aryl group and the heteroaryl group is bonded to a nitrogen atom.

According to one embodiment of the present invention, preferably, in Formula 1, A is selected from the group consisting of a substituted or unsubstituted aryl group having 6 to 30 carbon atoms and a substituted or unsubstituted heteroaryl group having 3 to 30 carbon atoms,
L1 and L2 are the same as or different from each other, and are each independently selected from the group consisting of a single bond and a substituted or unsubstituted arylene group having 6 to 30 carbon atoms,
n1 and n2 are respectively 1 or 2,
Rs are the same as or different from each other, and are each independently selected from the group consisting of a hydrogen atom, a deuterium atom, a substituted or unsubstituted alkyl group having 1 to 8 carbon atoms, a substituted or unsubstituted aryl group having 6 to 30 carbon atoms, and a substituted or unsubstituted heteroaryl group having 3 to 30 carbon atoms.
R1 to R8 are the same as or different from each other, and are each independently selected from the group consisting of a hydrogen atom, a deuterium atom, a substituted or unsubstituted alkyl group having 1 to 8 carbon atoms, a substituted or unsubstituted aryl group having 6 to 18 carbon atoms, and a substituted or unsubstituted heteroaryl group having 3 to 18 carbon atoms.

According to one embodiment of the present invention, the host may have a molecular weight of 450 or more, preferably 500 or more, more preferably 550 or more, and even more preferably 600 or more.

According to one embodiment of the present invention, the host may comprise at least one alkyl group having 1 to 8 carbon atoms.

According to one embodiment of the present invention, the host may comprise at least one deuterium.

According to one embodiment of the present invention, the preferred compound of the host is any one of following Compounds 1 to 102.

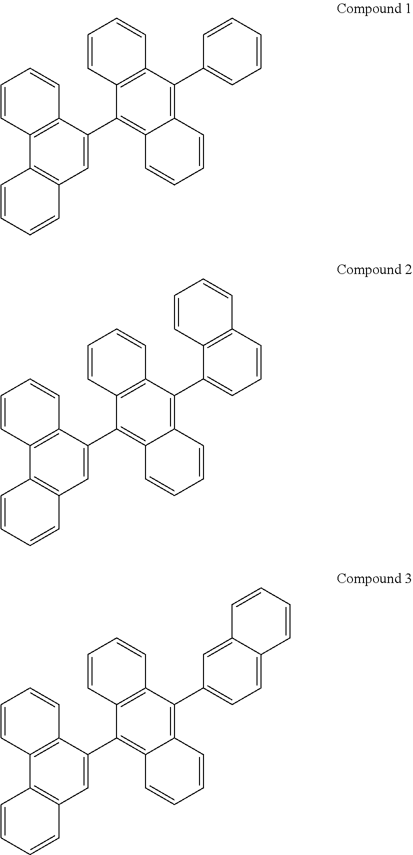

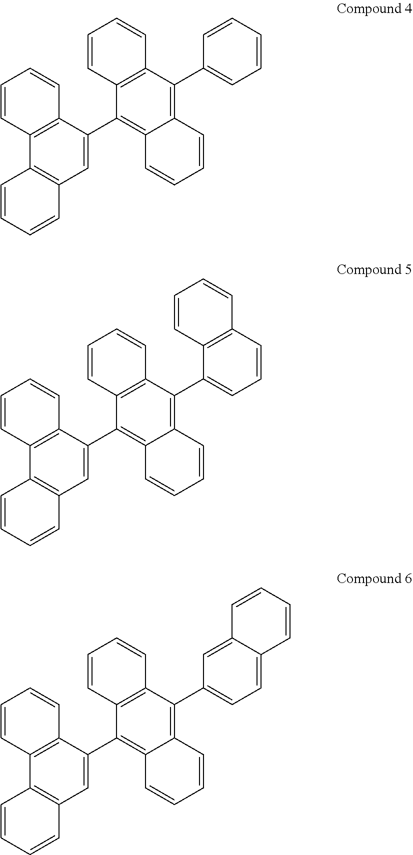

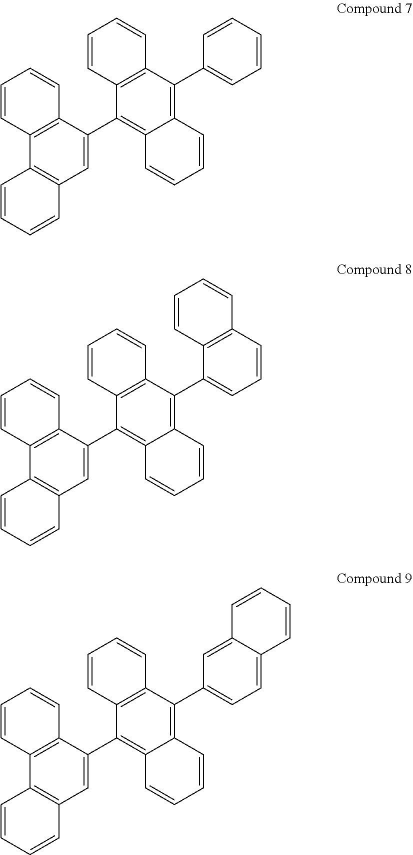

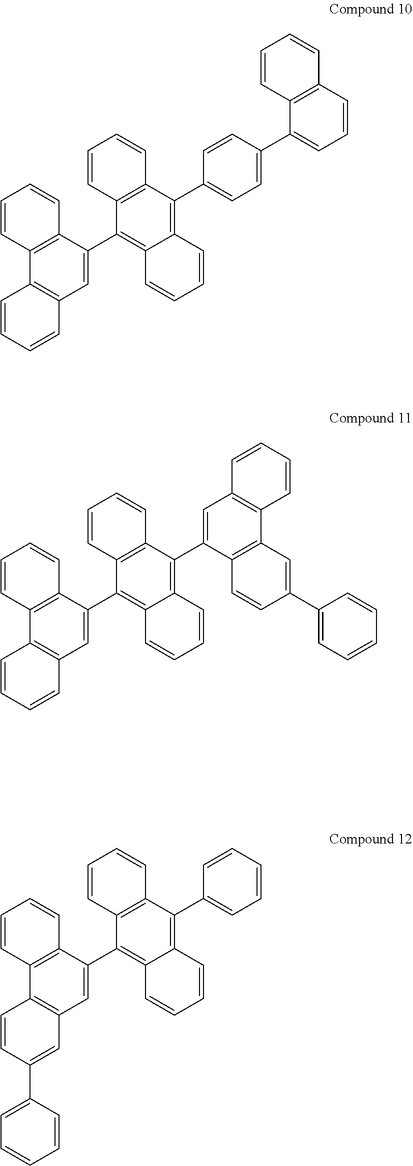

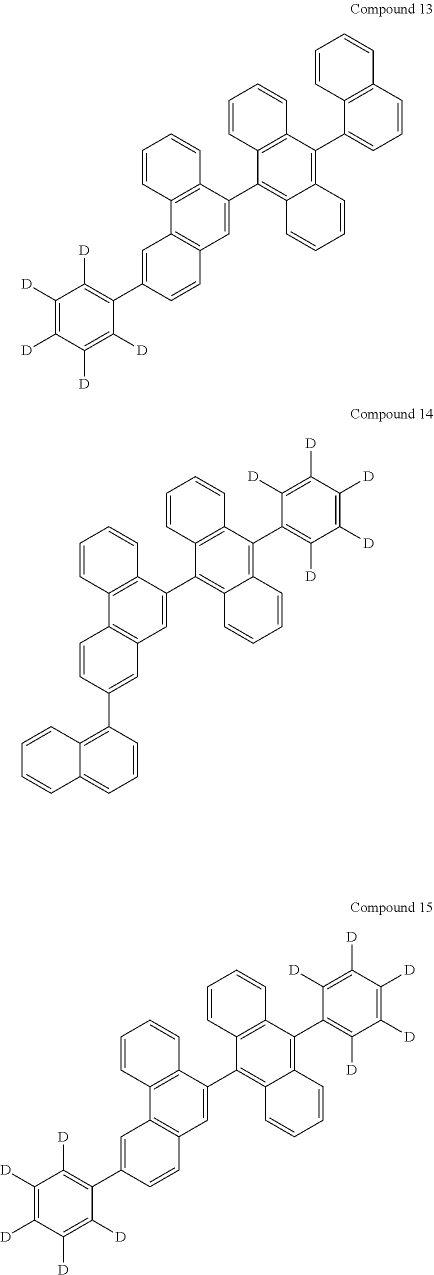 [SMILES: [2H]c1c([2H])c([2H])c(-c2ccc3cc(-c4c5ccccc5c(-c5cccc6ccccc56)c5ccccc45)c4ccccc4c3c2)c([2H])c1[2H] ; [2H]c1c([2H])c([2H])c(-c2c3ccccc3c(-c3cc4cc(-c5cccc6ccccc56)ccc4c4ccccc34)c3ccccc23)c([2H])c1[2H] ; [2H]c1c([2H])c([2H])c(-c2ccc3cc(-c4c5ccccc5c(-c5c([2H])c([2H])c([2H])c([2H])c5[2H])c5ccccc45)c4ccccc4c3c2)c([2H])c1[2H]]

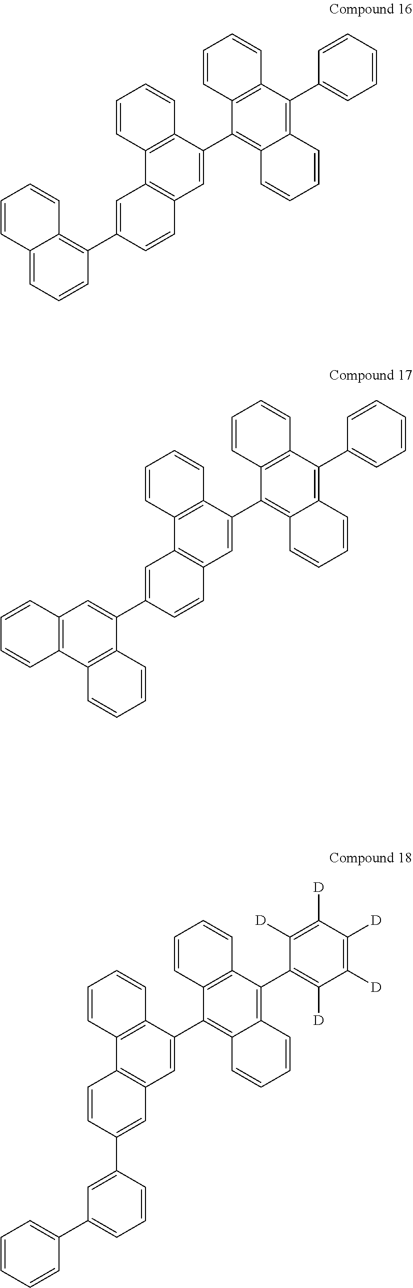

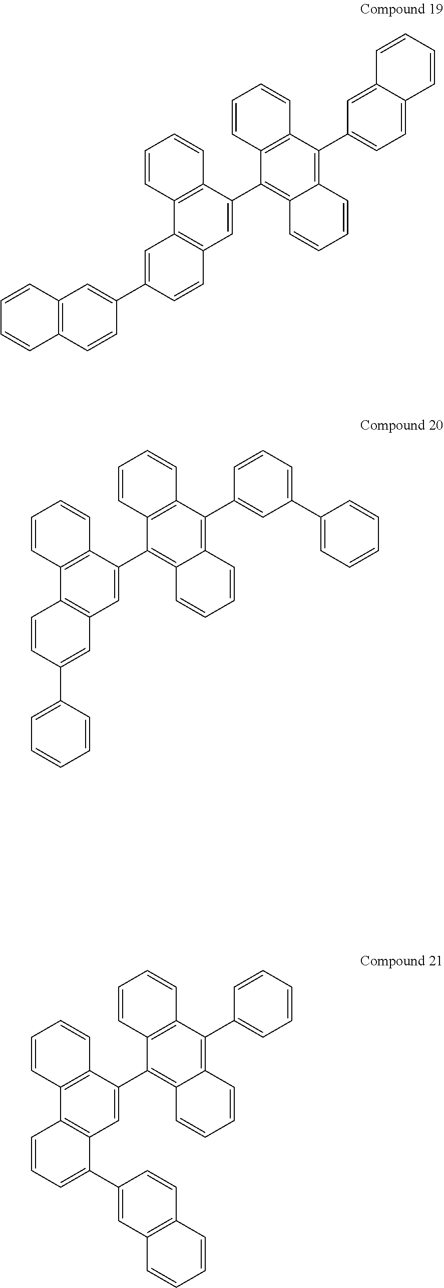

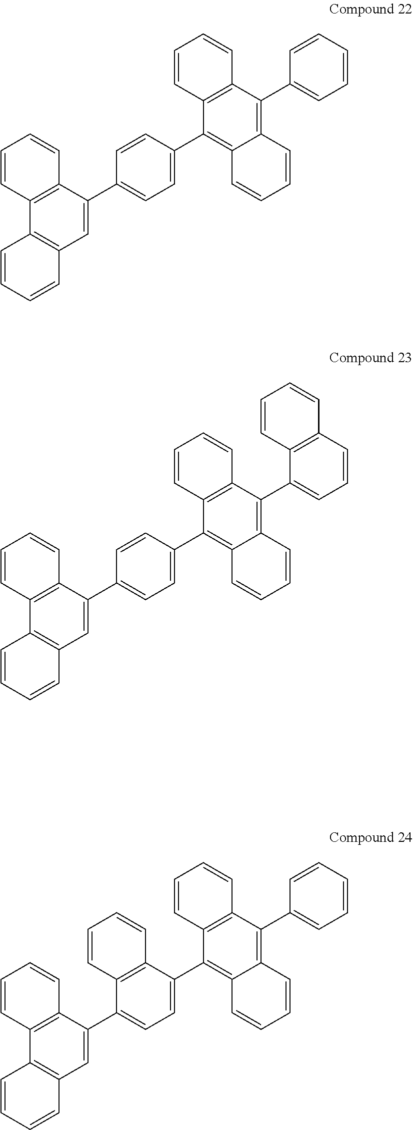

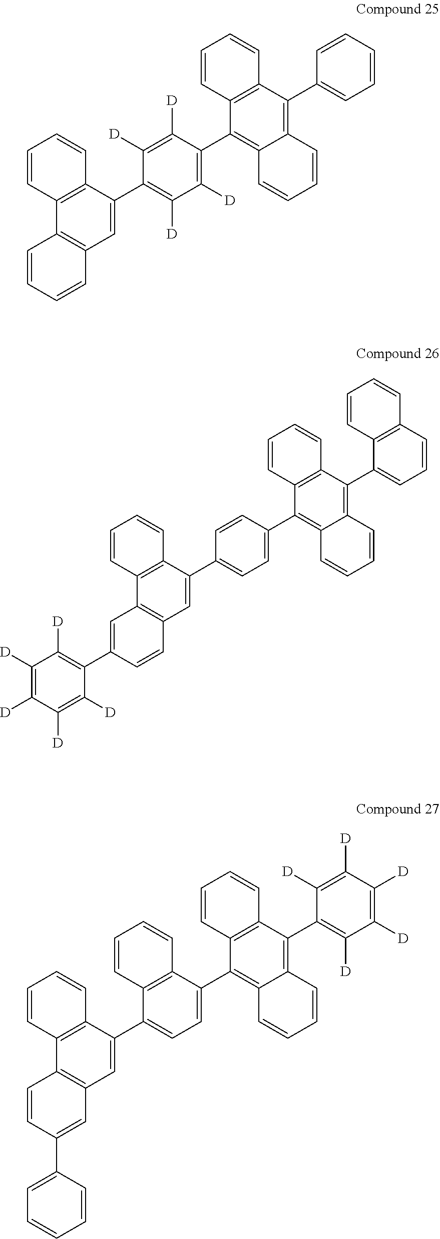 [SMILES: [2H]c1c([2H])c(-c2cc3ccccc3c3ccccc23)c([2H])c([2H])c1-c1c2ccccc2c(-c2ccccc2)c2ccccc12 ; [2H]c1c([2H])c([2H])c(-c2ccc3cc(-c4ccc(-c5c6ccccc6c(-c6cccc7ccccc67)c6ccccc56)cc4)c4ccccc4c3c2)c([2H])c1[2H] ; [2H]c1c([2H])c([2H])c(-c2c3ccccc3c(-c3ccc(-c4cc5cc(-c6ccccc6)ccc5c5ccccc45)c4ccccc34)c3ccccc23)c([2H])c1[2H]]

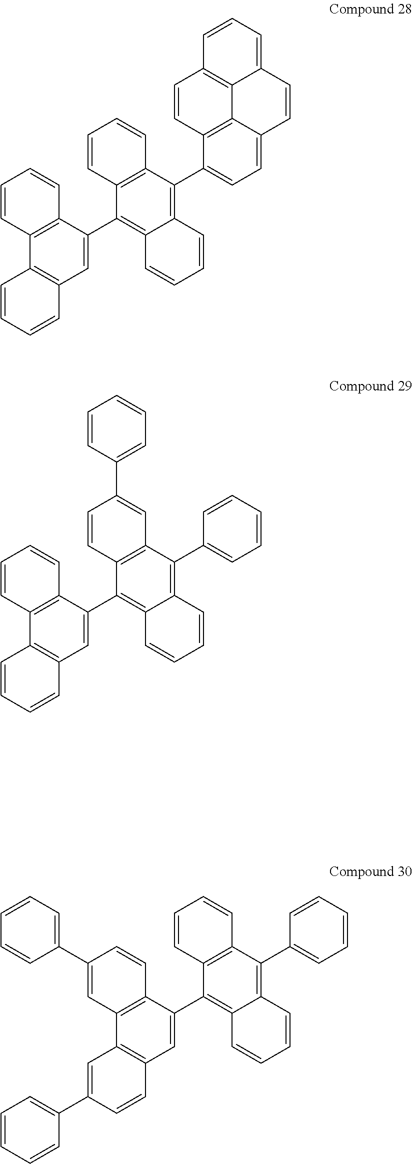

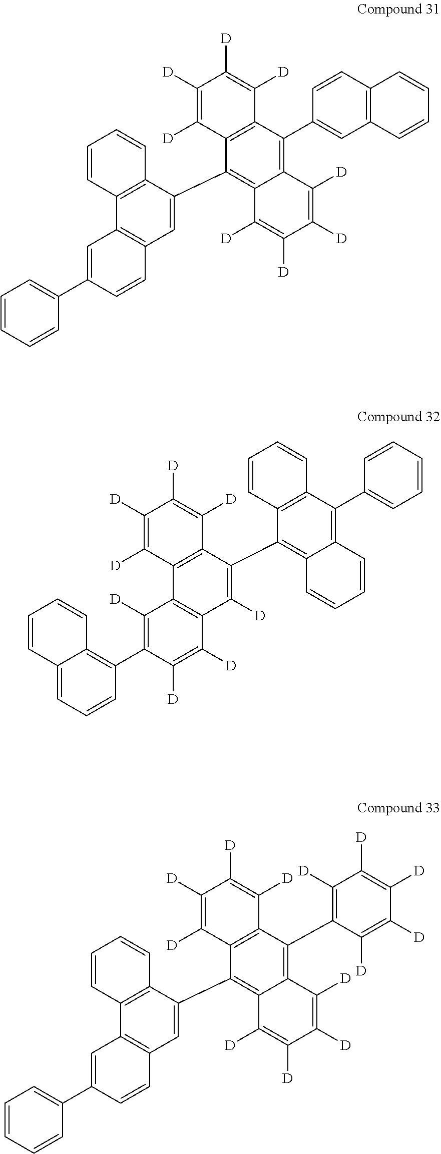 [SMILES: [2H]c1c([2H])c([2H])c2c(-c3cc4ccc(-c5ccccc5)cc4c4ccccc34)c3c([2H])c([2H])c([2H])c([2H])c3c(-c3ccc4ccccc4c3)c2c1[2H] ; [2H]c1c(-c2cccc3ccccc23)c([2H])c2c(c1[2H])c([2H])c(-c1c3ccccc3c(-c3ccccc3)c3ccccc13)c1c([2H])c([2H])c([2H])c([2H])c12 ; [2H]c1c([2H])c([2H])c(-c2c3c([2H])c([2H])c([2H])c([2H])c3c(-c3cc4ccc(-c5ccccc5)cc4c4ccccc34)c3c([2H])c([2H])c([2H])c([2H])c23)c([2H])c1[2H]]

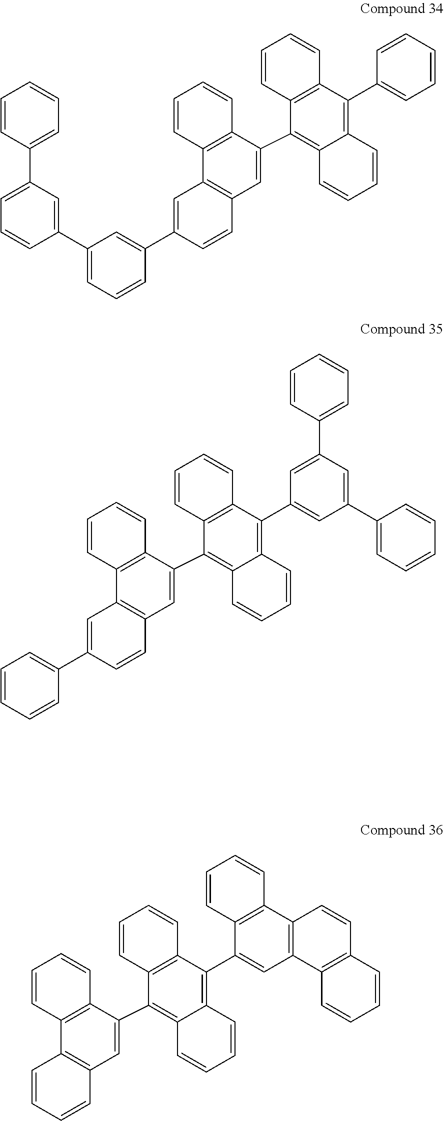

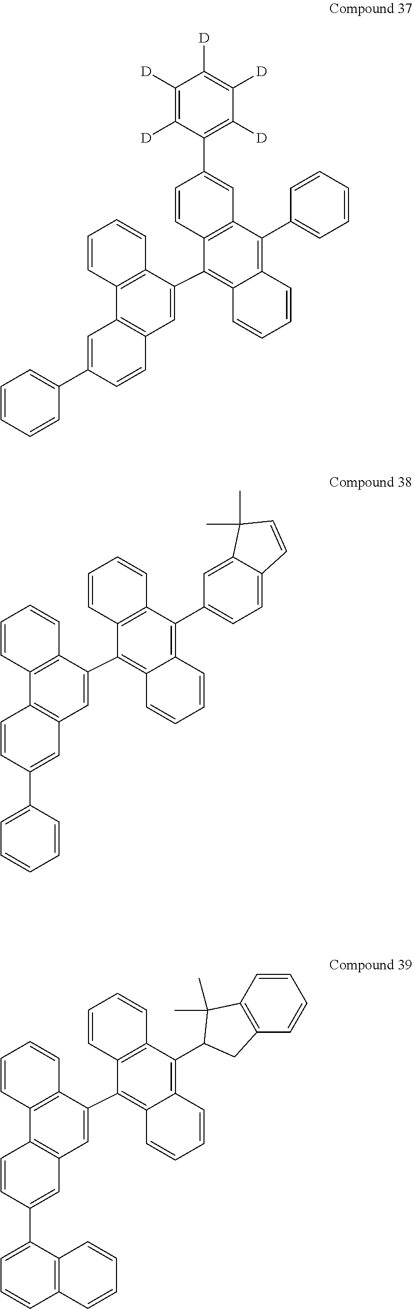

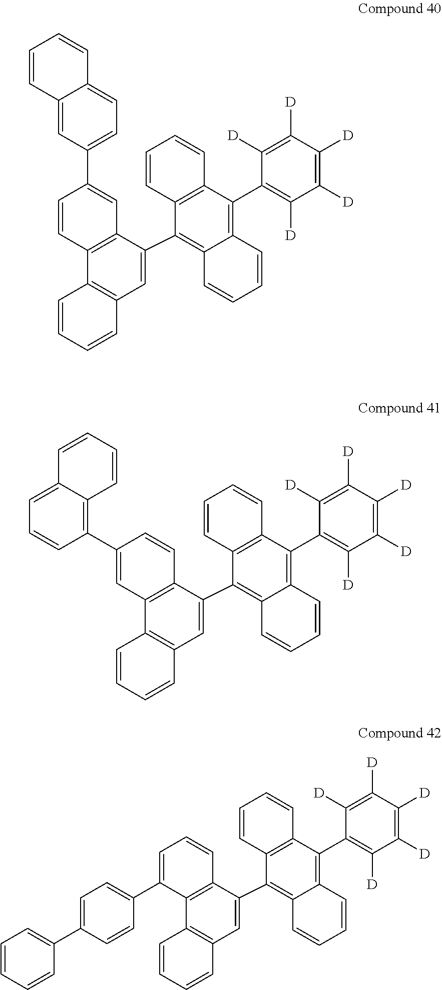 [SMILES: [2H]c1c([2H])c([2H])c(-c2c3ccccc3c(-c3cc4ccccc4c4ccc(-c5ccc6ccccc6c5)cc34)c3ccccc23)c([2H])c1[2H] ; [2H]c1c([2H])c([2H])c(-c2c3ccccc3c(-c3cc4ccccc4c4cc(-c5cccc6ccccc56)ccc34)c3ccccc23)c([2H])c1[2H] ; [2H]c1c([2H])c([2H])c(-c2c3ccccc3c(-c3cc4ccccc4c4c(-c5ccc(-c6ccccc6)cc5)cccc34)c3ccccc23)c([2H])c1[2H]]

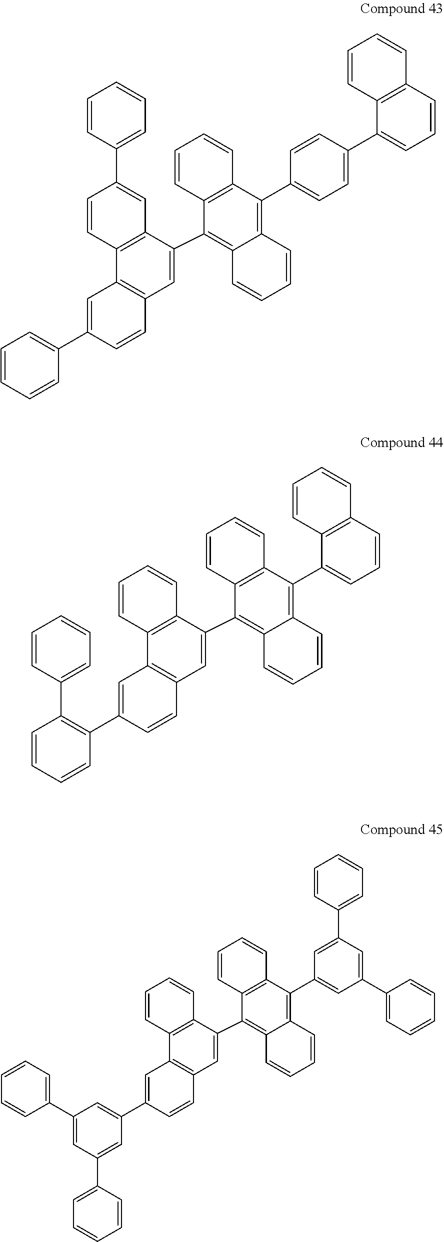

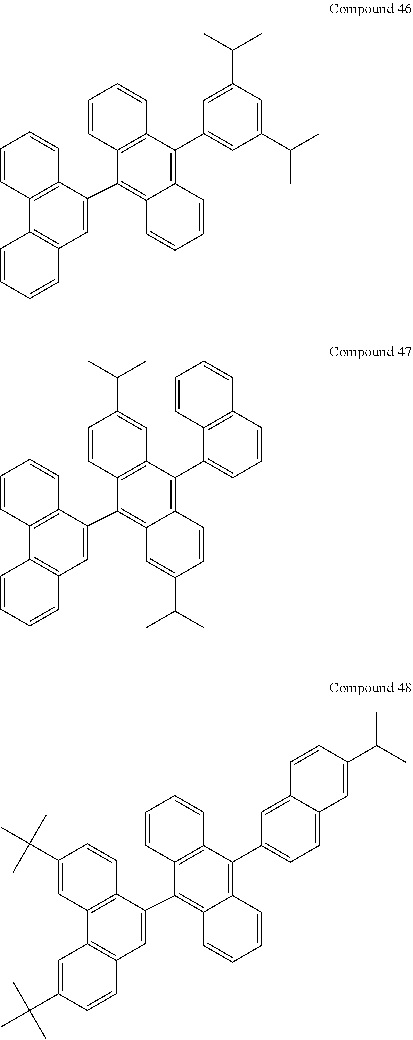

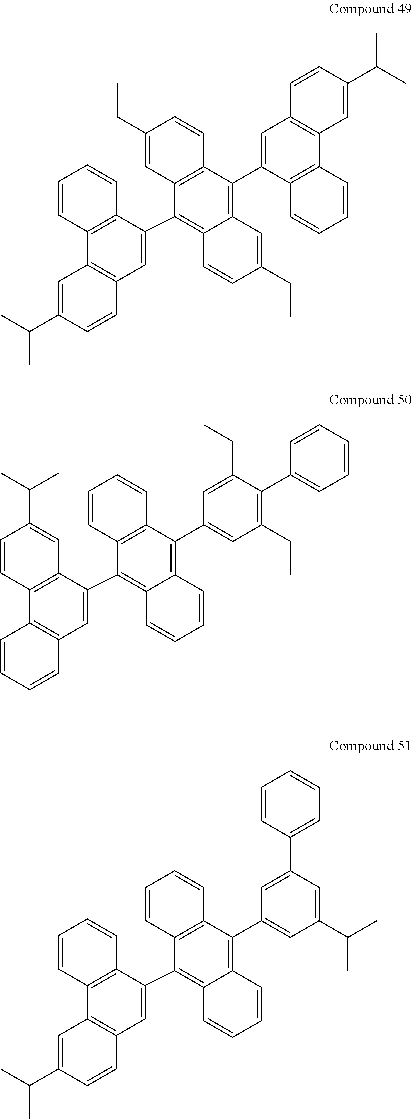

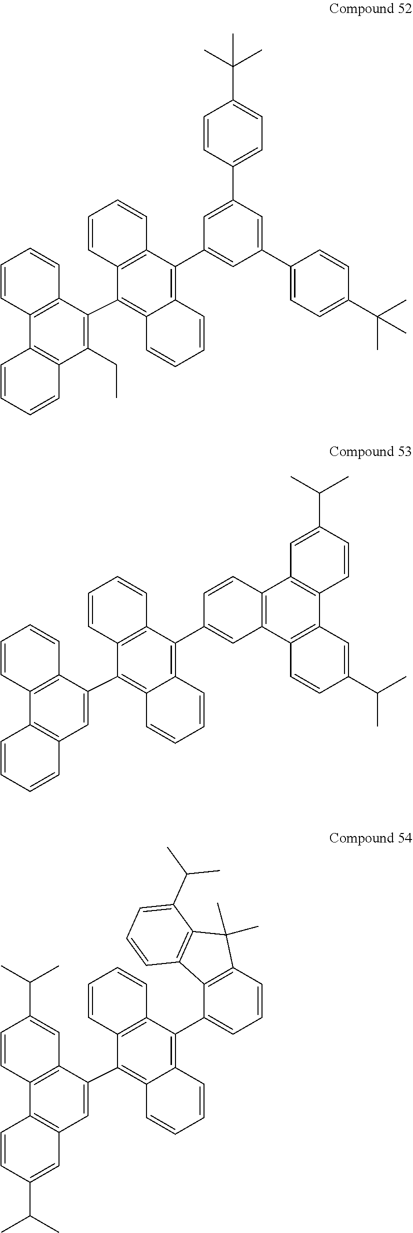

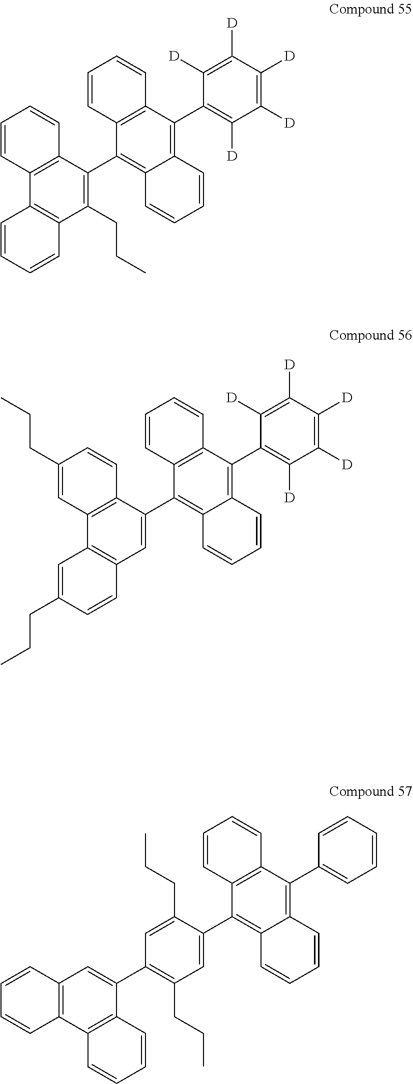

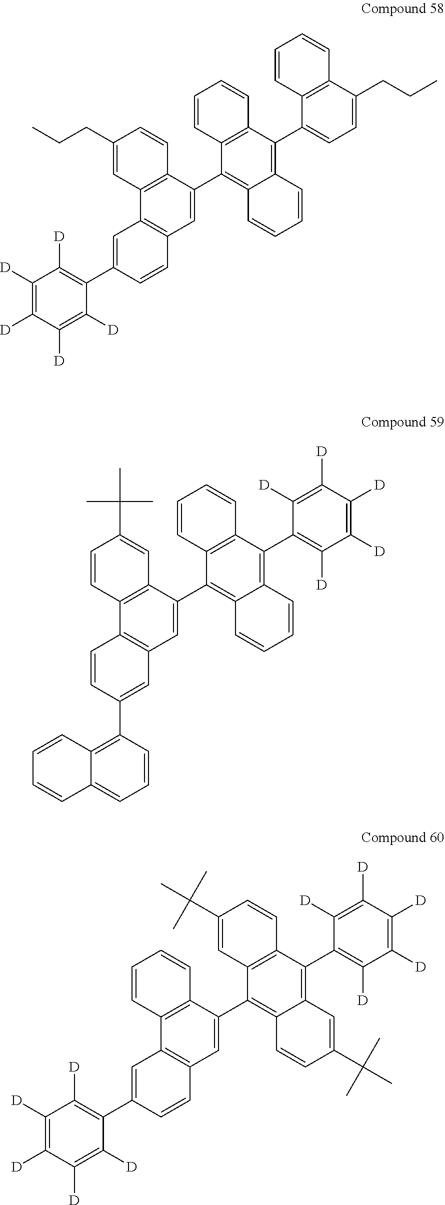 [SMILES: [2H]c1c([2H])c([2H])c(-c2ccc3cc(-c4c5ccccc5c(-c5ccc(CCC)c6ccccc56)c5ccccc45)c4ccc(CCC)cc4c3c2)c([2H])c1[2H] ; [2H]c1c([2H])c([2H])c(-c2c3ccccc3c(-c3cc4cc(-c5cccc6ccccc56)ccc4c4ccc(C(C)(C)C)cc34)c3ccccc23)c([2H])c1[2H] ; [2H]c1c([2H])c([2H])c(-c2ccc3cc(-c4c5ccc(C(C)(C)C)cc5c(-c5c([2H])c([2H])c([2H])c([2H])c5[2H])c5ccc(C(C)(C)C)cc45)c4ccccc4c3c2)c([2H])c1[2H]]

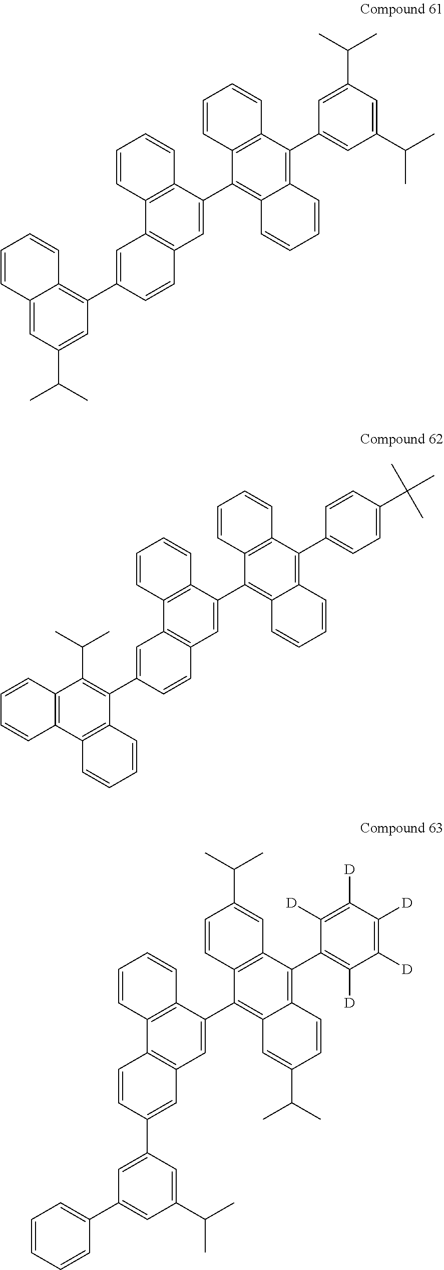

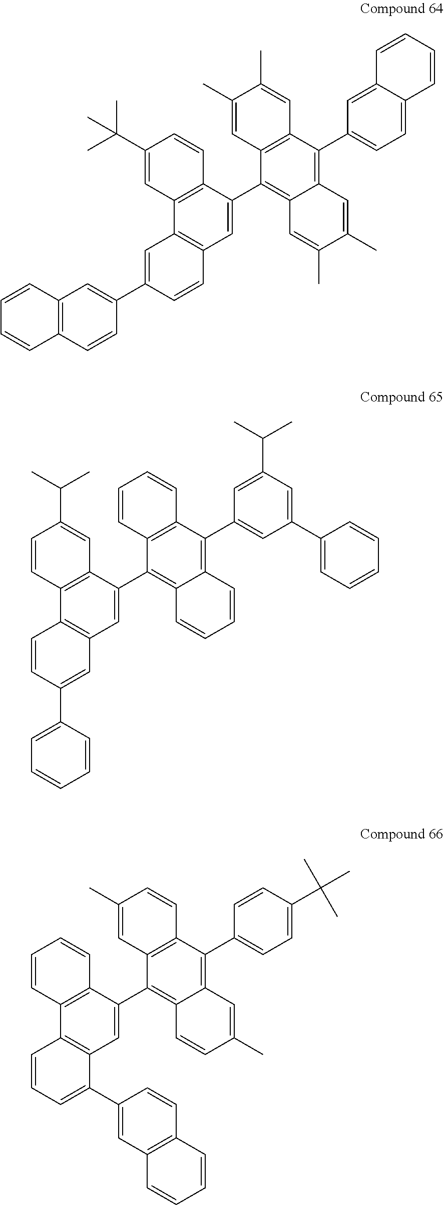

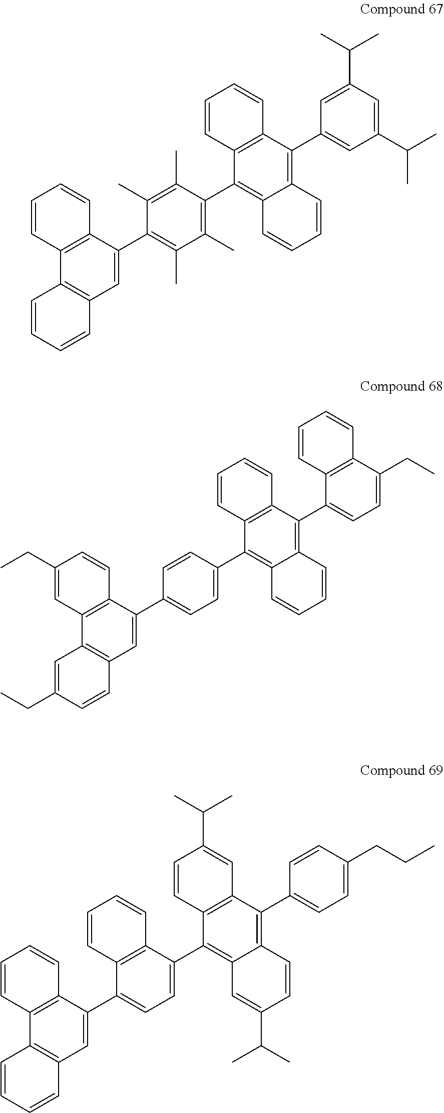

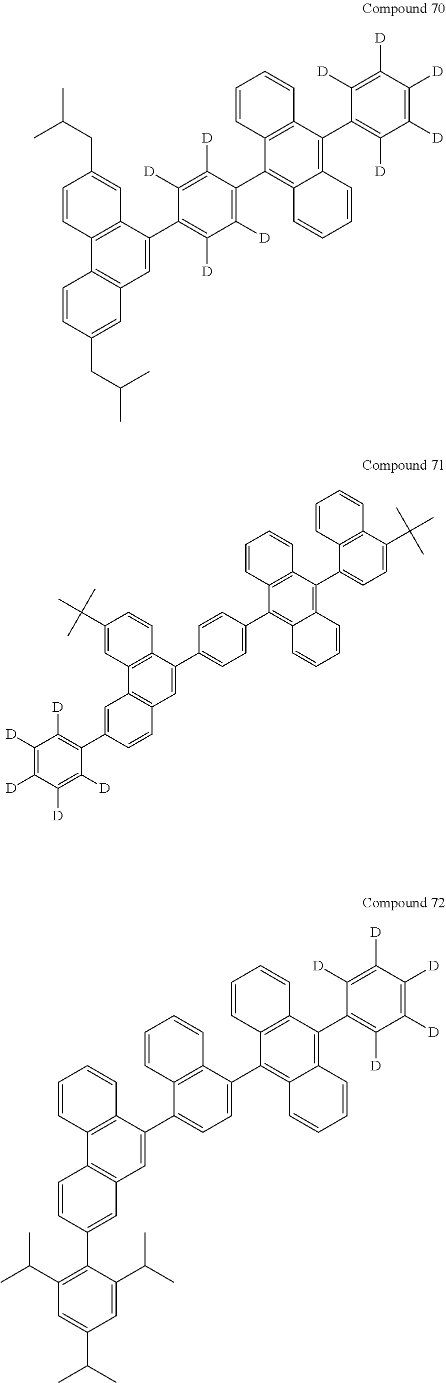

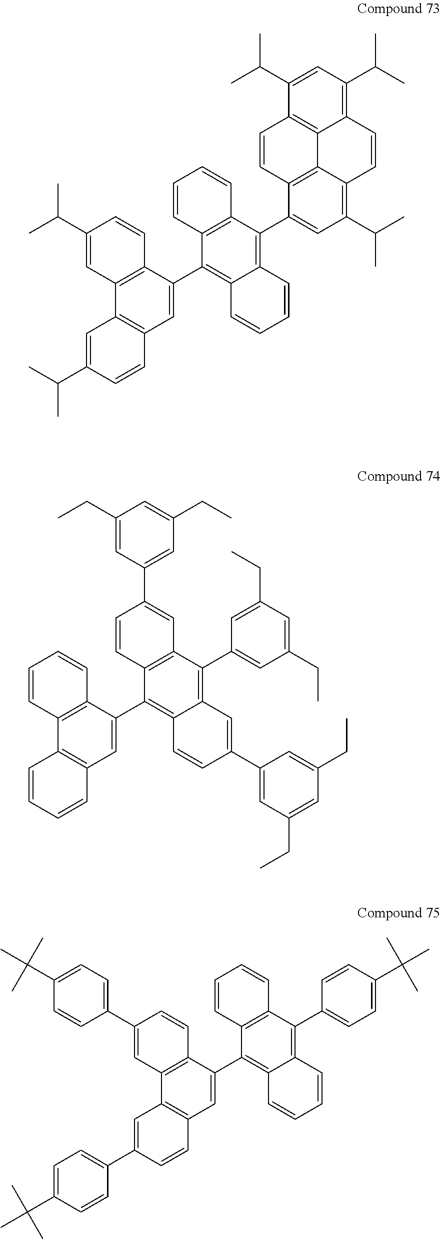

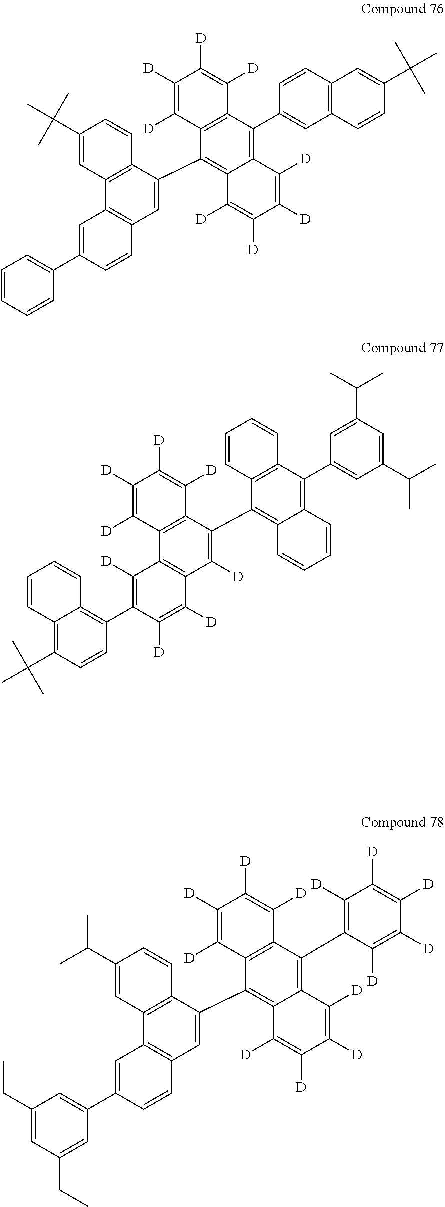

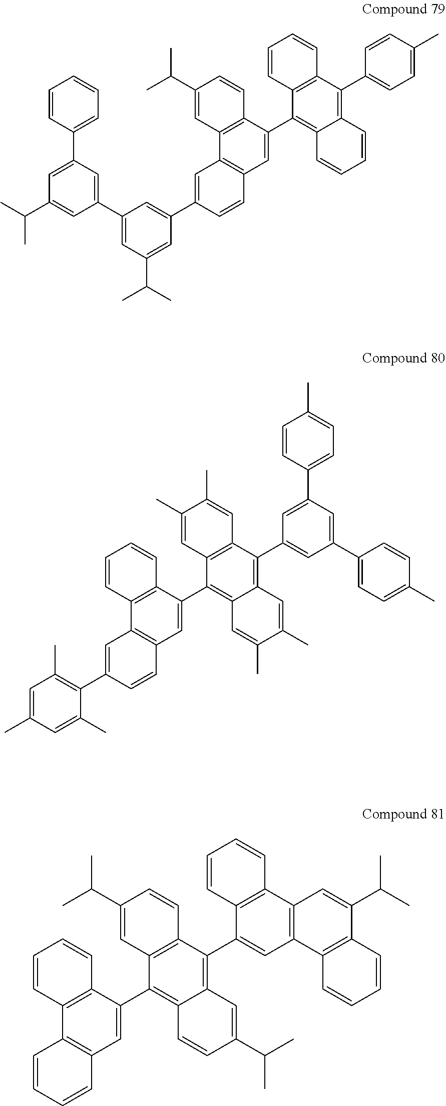

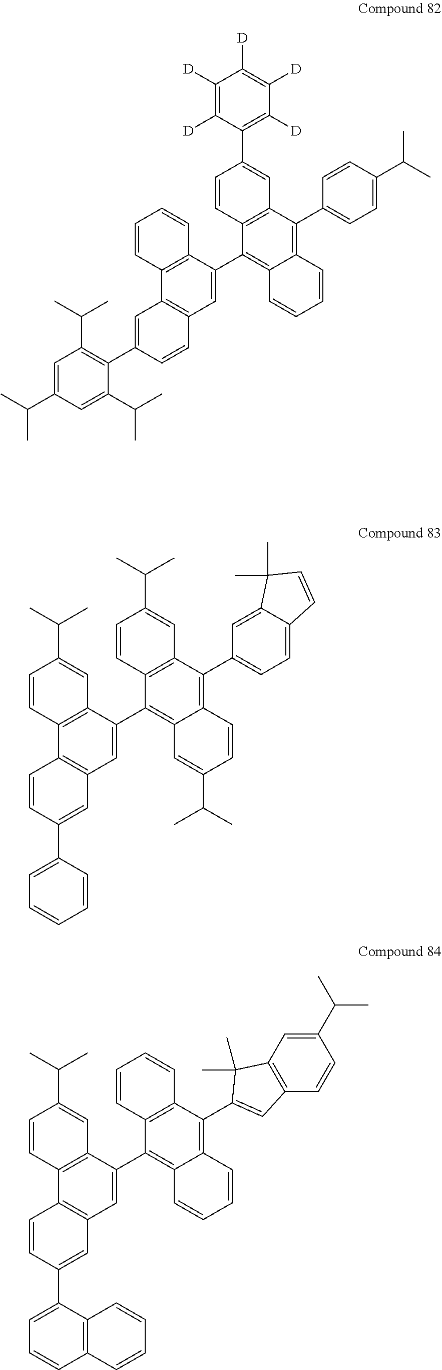

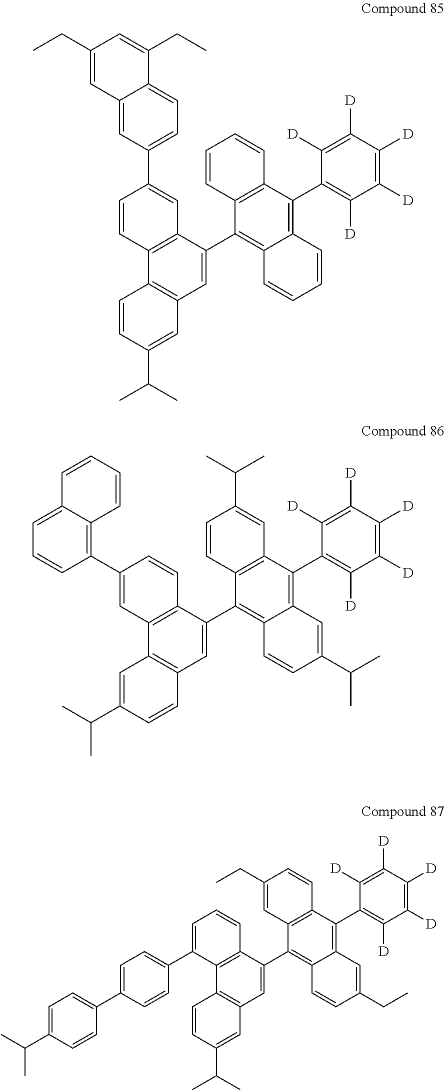 [SMILES: [2H]c1c([2H])c([2H])c(-c2c3ccccc3c(-c3cc4cc(C(C)C)ccc4c4ccc(-c5ccc6c(CC)cc(CC)cc6c5)cc34)c3ccccc23)c([2H])c1[2H] ; [2H]c1c([2H])c([2H])c(-c2c3cc(C(C)C)ccc3c(-c3cc4ccc(C(C)C)cc4c4cc(-c5cccc6ccccc56)ccc34)c3ccc(C(C)C)cc23)c([2H])c1[2H] ; [2H]c1c([2H])c([2H])c(-c2c3cc(CC)ccc3c(-c3cc4cc(C(C)C)ccc4c4c(-c5ccc(-c6ccc(C(C)C)cc6)cc5)cccc34)c3cc(CC)ccc23)c([2H])c1[2H]]

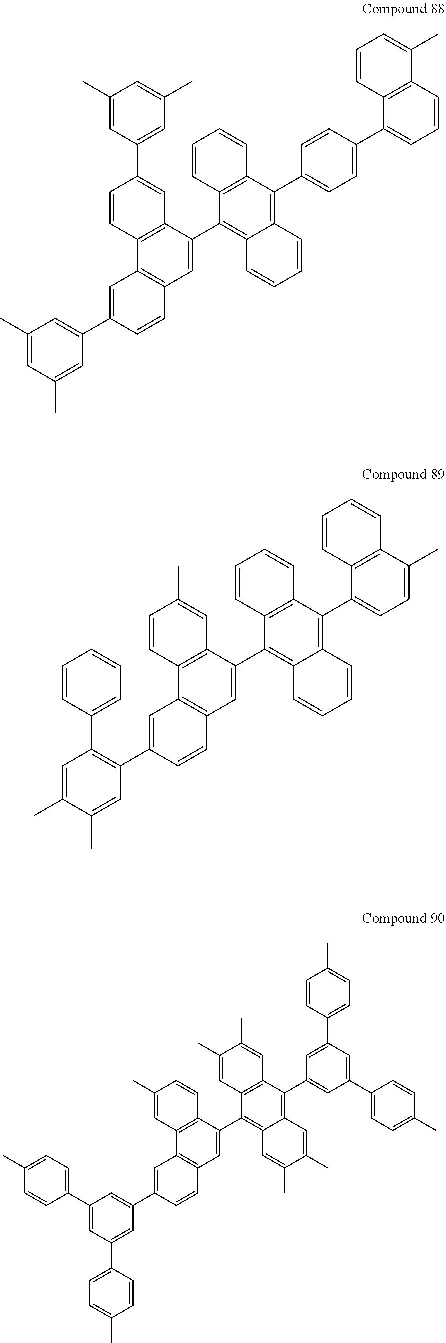

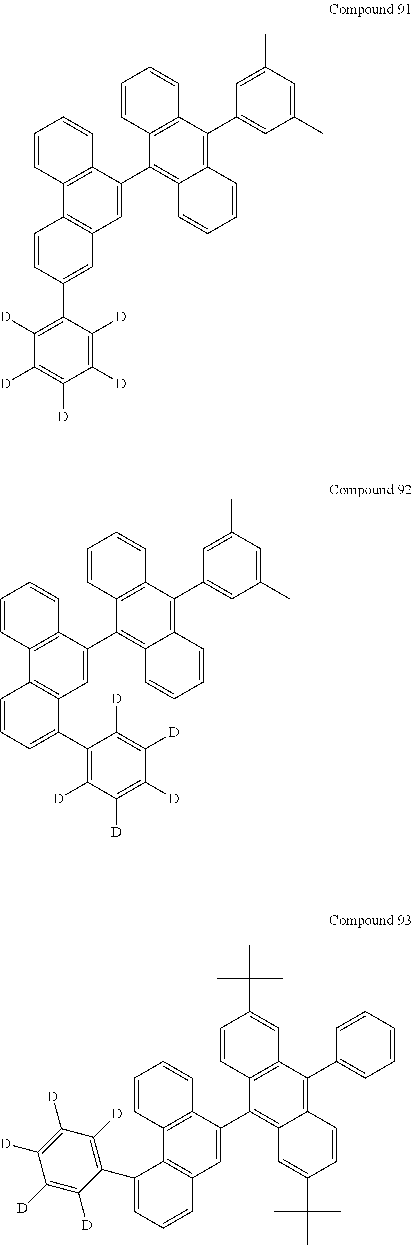 [SMILES: [2H]c1c([2H])c([2H])c(-c2ccc3c(c2)cc(-c2c4ccccc4c(-c4cc(C)cc(C)c4)c4ccccc24)c2ccccc23)c([2H])c1[2H] ; [2H]c1c([2H])c([2H])c(-c2cccc3c2cc(-c2c4ccccc4c(-c4cc(C)cc(C)c4)c4ccccc24)c2ccccc23)c([2H])c1[2H] ; [2H]c1c([2H])c([2H])c(-c2cccc3cc(-c4c5ccc(C(C)(C)C)cc5c(-c5ccccc5)c5ccc(C(C)(C)C)cc45)c4ccccc4c23)c([2H])c1[2H]]

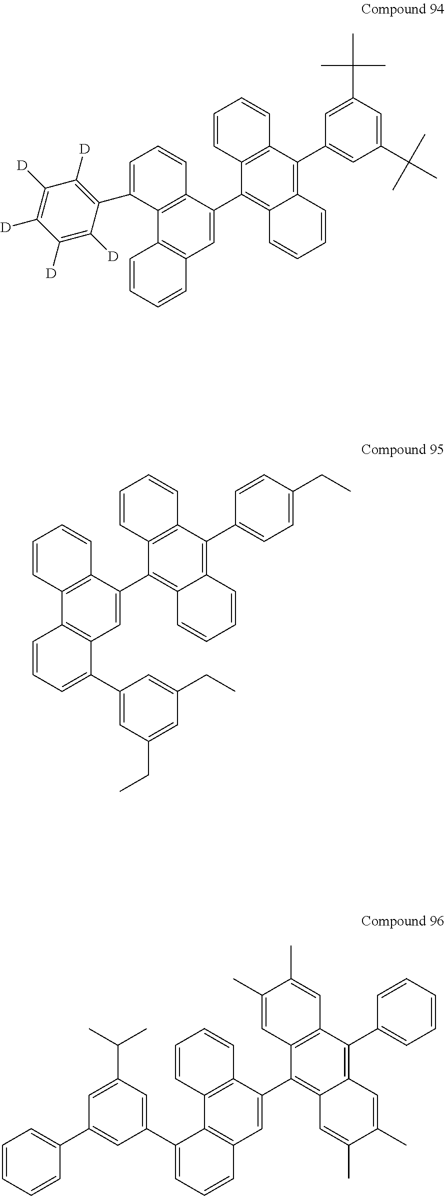

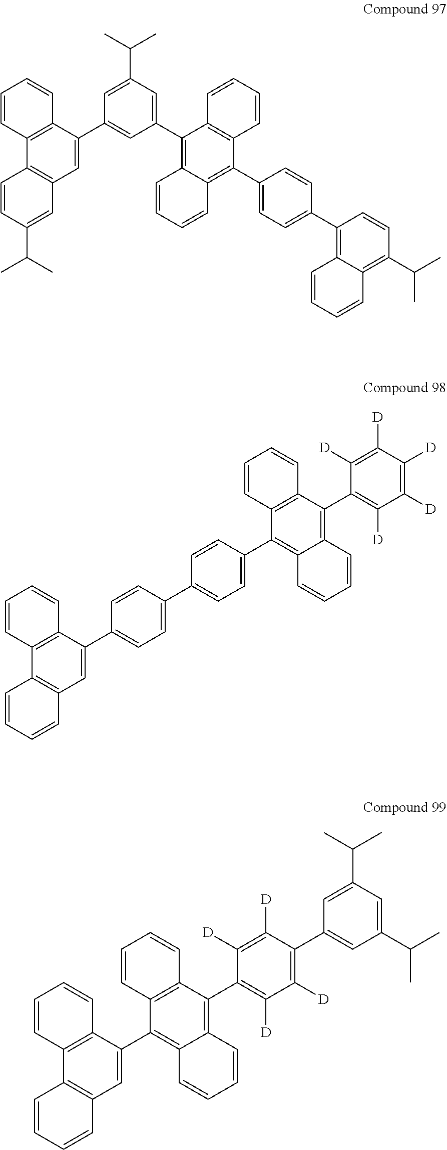

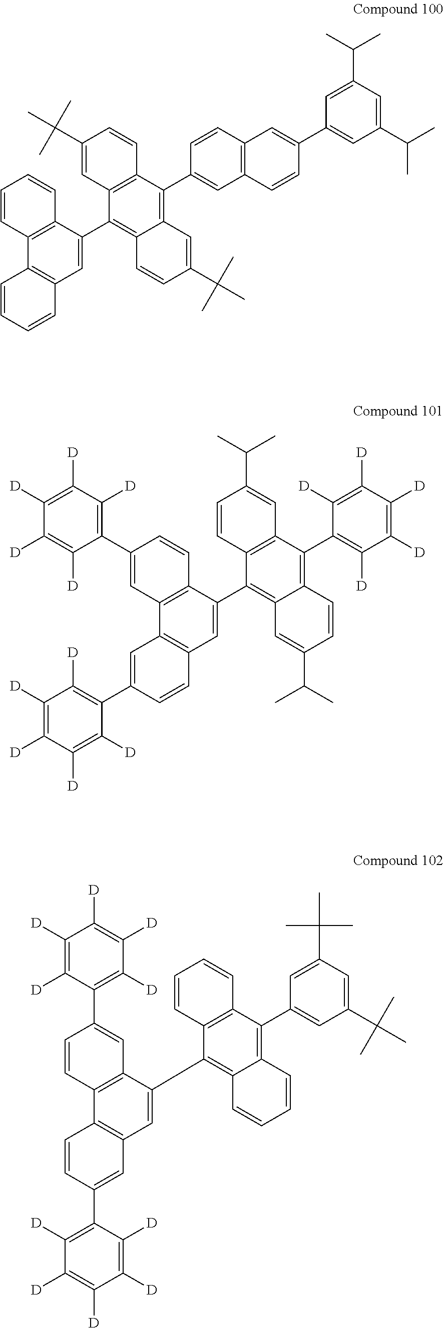

The compound represented by Formula 1 may have a solubility of 0.1 wt % to 50 wt %, and preferably 0.5 wt % to 20 wt % with respect to an organic solvent, but is not limited thereto.

The organic solvent may include at least one of a chlorine-based solvent, an ether-based solvent, an aromatic solvent, an aliphatic solvent, a ketone-based solvent, an ester-based solvent, an alcohol-based solvent, an amide-based solvent, and a benzoate-based solvent. The solvent may be a single pure material or a mixture, and may preferably be a benzoate-based solvent.

For example, the chlorine-based solvent may include chloroform, methylene chloride, or chlorobenzene, the ether-based solvent may include tetrahydrofuran or dioxane, the aromatic solvent may include toluene, xylene, or trimethylbenzene, the aliphatic solvent may include cyclohexane, n-pentane, or n-hexane, the ketone-based solvent may include acetone, methyl ethyl ketone, or cyclohexanone, the ester-based solvent may include ethyl acetate or butyl acetate, the alcohol-based solvent may include methanol, ethanol, propanol, or cyclohexanol, the amide-based solvent may include N,N-dimethylformamide, and the benzoate-based solvent may include methyl benzoate, ethyl benzoate, or butyl benzoate.

The organic solvent may be used alone or in a mixture with two or more solvents.

The boiling point of the organic solvent may be 60° C. to 300° C., and preferably 130° C. to 300° C., but is not limited thereto.

The viscosity of the organic solvent may be 1 cP to 10 cP, and preferably 2 cP to 8 cP, but is not limited thereto.

The composition comprising the compound represented by Formula 1 and the organic solvent is suitable for the manufacture of an organic light emitting device using a solution process.

The composition may further comprise a fluorescent dopant or a phosphorescent dopant.

The fluorescent dopant may include, for example, a pyrene-based compound, a deuterium-substituted pyrene-based compound, an arylamine, a deuterium-substituted arylamine, a peryl-based compound, a deuterium-substituted peryl-based compound, a pyrrole-based compound, a deuterium-substituted pyrrole-based compound, a boron-based compound, a fluorene-based compound, a deuterium-substituted fluorene-based compound, a hydrazone-based compound, a deuterium-substituted hydrazone-based compound, a carbazole-based compound, a deuterium-substituted carbazole-based compound, a stilbene-based compound, a deuterium-substituted stilbene-based compound, a starburst-based compound, a deuterium-substituted starburst-based compound, an oxadiazole-based compound, a deuterium-substituted oxadiazole-based compound, coumarine, or a deuterium-substituted coumarine, but may not be limited thereto.

The phosphorescent dopant may be, for example, an organometallic compound including iridium, platinum, osmium, titanium, zirconium, hafnium, europium, terbium, thulium, iron, cobalt, nickel, ruthenium, rhodium, palladium, or a combination thereof, but is not limited thereto.

The fluorescent dopant or the phosphorescent dopant may be 0.01 to 20 parts by weight based on 100 parts by weight of the host.

The content of the organic electroluminescent material comprising the compound represented by Formula 1 in the composition may be 0.5 wt % or more, and preferably 1.0 wt % or more, but is not limited thereto.

Hereinafter, a method for manufacturing an organic electroluminescent device according to an embodiment of the present invention will be described with reference to FIG. 1.

A substrate 10 may be prepared. The substrate 10 may be an organic substrate or a transparent plastic substrate having excellent transparency, surface smoothness, ease of handling, and water resistance, but is not limited thereto, and may include a substrate generally used in an organic electroluminescent device.

The first electrode 20 may be formed by coating an anode material for electrode on the top surface of the substrate 10. The anode material for electrode may include a transparent and highly conductive material, for example, indium tin oxide (ITO), indium zinc oxide (IZO), tin oxide (SnO2), and/or zinc oxide (ZnO).

The hole injecting layer 30 may be formed on the top surface of the first electrode 20 by vacuum thermal depositing or spin coating a hole injecting layer material. The hole transport layer 40 may be formed on the top surface of the first electrode 20 by vacuum thermal depositing or spin coating a hole injecting layer material.

An electron blocking layer (not shown) may be selectively formed on the top surface of the hole transport layer 40 by vacuum thermal depositing or spin coating an electron blocking layer material. The electron blocking layer prevents electrons, injected from the electron injecting layer 70, from passing through the organic light emitting layer 50 and entering the hole transport layer 40, thereby improving the lifespan and efficiency of the device. The electron blocking layer may be formed at an appropriate portion between the organic light emitting layer 50 and the hole injecting layer 30, and preferably, between the organic light emitting layer 50 and the hole transport layer 40.

The organic light emitting layer 50 may be formed on the top surface of the hole transport layer 40 or the electron blocking layer. The organic light emitting layer 50 may be formed using a solution containing the organic electroluminescent material and the solvent. More specifically, the organic light emitting layer 50 may be formed by coating the solution on the top surface of the hole transport layer 40 by any one of a spin coating method, a dip coating method, a doctor blade coating method, a spray coating method, a roll coating method, an inkjet printing method, and a screen printing method.

According to an embodiment of the present invention, the organic light emitting layer 50 may have a thickness of 50 Å to 2,000 Å.

A hole blocking layer (not shown) may be selectively formed on the top surface of the organic light emitting layer 50 by a vacuum deposition method or a spin coating method. The hole blocking layer may include a hole blocking material having a very low HOMO (Highest Occupied Molecular Orbital) level to prevent holes from passing through the organic light emitting layer 50 and flowing into the second electrode 80. This is because the lifespan and efficiency of the organic electroluminescent device are reduced when the holes are introduced into the second electrode 80 through the organic light emitting layer 50. The hole blocking material may have an electron transport capability while having an ionization potential higher than that of an organic electroluminescent material, but is not particularly limited. The hole blocking material may include, for example, BAlq, BCP, TPBI, etc.

The electron transport layer 60 may be deposited on the top surface of the organic light emitting layer 50 or the hole blocking layer by a vacuum deposition method or a spin coating method to form the electron injection layer 70, and the second electrode 80 may be formed on the top surface of the electron injection layer 70 by vacuum thermal depositing a metal for a cathode electrode. The metal for a cathode electrode may include, for example, lithium (Li), magnesium (Mg), aluminum (Al), aluminum-lithium (Al—Li), calcium (Ca), magnesium-indium (Mg—In), magnesium-silver (Mg—Ag), or the like. When a top emission organic electroluminescent device is manufactured, the metal for a cathode electrode may include indium tin oxide (ITO) or indium zinc oxide (IZO).

An organic electroluminescent device according to an embodiment of the present invention may be manufactured by the above-described manufacturing method.

### EMBODIMENTS FOR CARRYING OUT THE INVENTION

The present invention is not limited by the following examples.

### Synthesis Example 1. Synthesis of <Compound 46>

### Synthesis Example 1-1. Synthesis of <1-a>

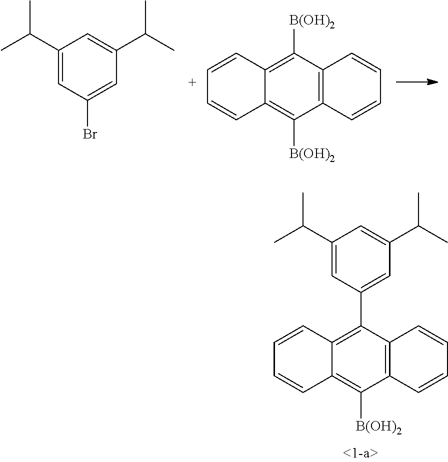

Nitrogen was purged in a 3000 ml round-bottomed flask, 7.24 g (0.03 mol) of 1-bromo-3,5-bis(1-methylethyl)benzene, 7.44 g (0.028 mol) of B,B′-9,10-anthracenediylbis[boronic acid], 0.64 g (0.0006 mol) of tetrakistriphenylphosphine palladium (Pd[PPh3]4), 9.00 g (0.065 mol) of potassium carbonate, 100 ml of toluene, and 30 ml of water were added thereto, and the mixture was refluxed for 12 hours. After the reaction was completed, the reaction solution was cooled to room temperature, layer separation was performed, and the organic layer was concentrated, and then separated using column chromatography to obtain <1-a>4.15 g (yield: 38.8%) through recrystallization.

### Synthesis Example 1-2. Synthesis of <Compound 46>

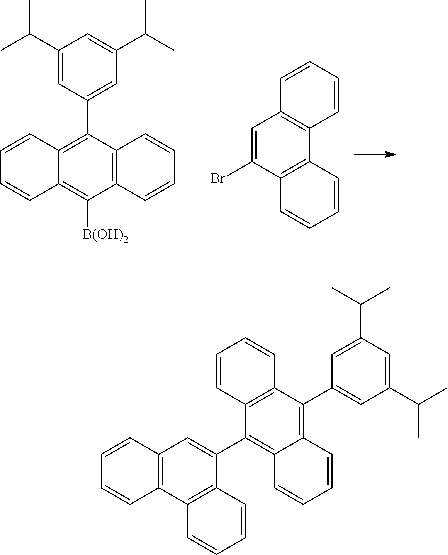

<Compound 46>

The synthesis was performed in the same manner as in Synthesis Example 1-1, except that 9-bromophenanthrene was used instead of <1-a>, 1-bromo-3,5-diethylbenzene instead of B,B′-9,10-anthracenediylbis[boronic acid], to obtain 10.2 g of <Compound 46>(yield: 70.8%).

MS(MALDI-TOF): m/z 514.27[M]

### Synthesis Example 2. Synthesis of <Compound 55>

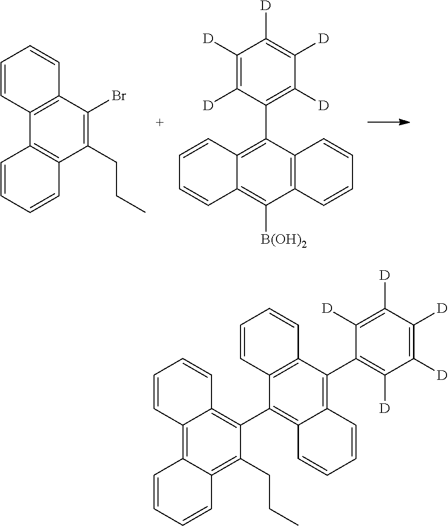

<Compound 55>

The synthesis was performed in the same manner as in Synthesis Example 1-2, except that 9-bromo-10-propylphenanthrene was used instead of 1,6-dibromo-3,8-bis(1-methylethyl)pyrene, to obtain 10.3 g of <Compound 55>(yield: 72.0%).

MS(MALDI-TOF): m/z 477.25[M]

### Test Examples

Solubility Evaluation

The solubility of each of the compound prepared in the synthesis examples and the comparative compound [BH1] was measured at room temperature in methyl benzoate and ethyl benzoate. The case in which all the components were dissolved at a concentration of 2 wt % was evaluated as O, and the case in which all the components were not dissolved was evaluated as X, and the results are shown in the following Table 1.

TABLE 1

| | Compound 46 | Compound 55 | BH1 |
|---|---|---|---|
| methyl benzoate | ◯ | ◯ | X |
| ethyl benzoate | ◯ | ◯ | X |

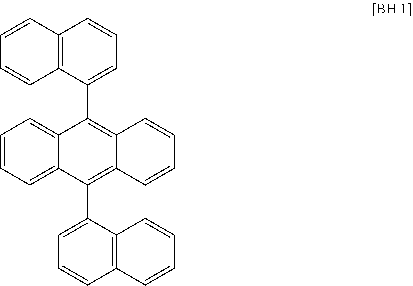 [SMILES: c1ccc2c(-c3c4ccccc4c(-c4cccc5ccccc45)c4ccccc34)cccc2c1]

### Examples 1 to 2

A hole injecting layer was formed by spin coating poly(3, 4-ethylenedioxythiophene): poly(styrene sulfonate) (AI4083), which has been widely used as a hole injecting layer, on the ITO transparent electrode to form a film having a thickness of 60 nm, and then baking it at 200° C. for 30 minutes. A hole transport layer was formed by spin coating TFB on the hole injecting layer and depositing to form a film having a thickness of 20 nm, and then baking it at 130° C. for 10 minutes. A light emitting layer was formed by spin coating a 2 wt % methyl benzoate solution containing the host compound according to the present invention and a dopant <BD>(3 wt %) as described in the following Table 2 on the hole transport layer to form a film with a thickness of 30 nm, and then baking at 180° C. for 30 minutes. This was baked at 130° C. for 10 minutes under a nitrogen gas atmosphere, and then, [E-1] and [E-2] were deposited as an electron transporting layer at a ratio of (1:1) to form a film with a thickness of 25 nm. On the electron transport layer, [E-2] was deposited as an electron injection layer to form a film having a thickness of 1 nm. Finally, aluminum was deposited as a cathode on the electron injection layer with a thickness of 100 nm to manufacture an organic light emitting device. The emission characteristics of the organic light emitting device were measured at 10 mA/cm2.

In the case of the comparative example compound, it was not dissolved by 2 wt % with respect to methyl benzoate, thereby making it impossible to manufacture a device.

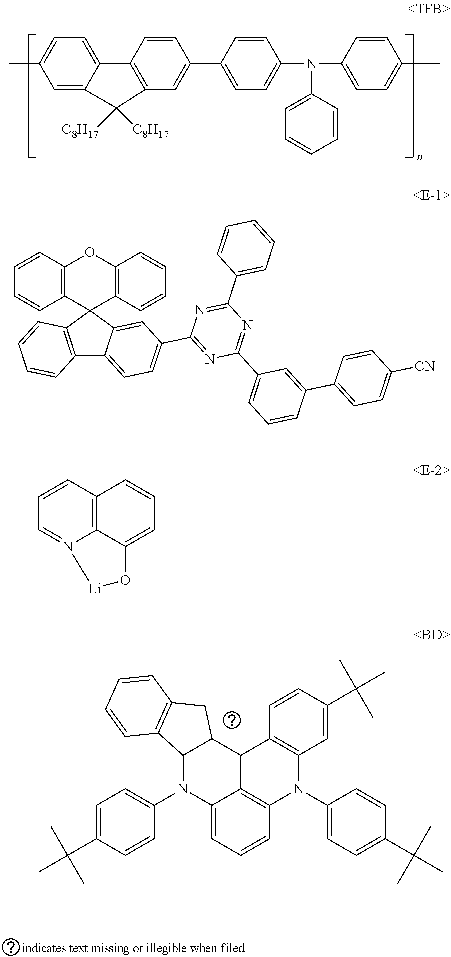

TABLE 2

| | | | Driving | |
|---|---|---|---|---|
| | Host | Dopant | voltage (V) | Efficiency (Cd/A) |
| Example 1 | Compound 46 | <BD> | 4.62 | 3.8 |
| Example 2 | Compound 55 | <BD> | 4.48 | 4.0 |

As shown in Table 1 and Table 2, it can be observed that the compound represented by Formula 1 of the present invention is suitable for a solution process, compared to the comparative compound [BH 1], and is applicable to an organic light emitting device.

1. An organic electroluminescent device, comprising:
a first electrode;
a second electrode; and
an organic material layer formed between the first electrode and the second electrode,
wherein the organic material layer is formed using a composition having an organic electroluminescent material and a solvent,
wherein the organic electroluminescent material has a host, and
wherein the host is one or more compounds represented by the following Formula 1: 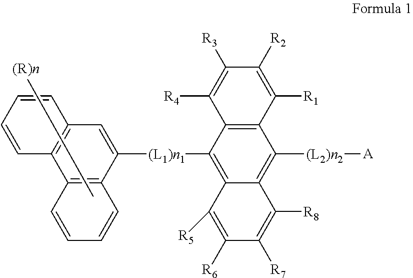
wherein in Formula 1, A is selected from the group consisting of a substituted or unsubstituted alkyl group having 1 to 30 carbon atoms, a substituted or unsubstituted alkenyl group having 2 to 30 carbon atoms, a substituted or unsubstituted cycloalkyl group having 3 to 30 carbon atoms, a substituted or unsubstituted cycloalkenyl group having 5 to 30 carbon atoms, a substituted or unsubstituted aryl group having 6 to 50 carbon atoms, and a substituted or unsubstituted heteroaryl group having 3 to 50 carbon atoms,
L1 and L2 are the same as or different from each other, and are each independently selected from the group consisting of a single bond, a substituted or unsubstituted arylene group having 6 to 50 carbon atoms, and a substituted or unsubstituted heteroarylene group having 3 to 50 carbon atoms,
n1 and n2 are respectively 1 or 2,
Rs are the same as or different from each other, and are each independently selected from the group consisting of a hydrogen atom, a deuterium atom, a substituted or unsubstituted alkyl group having 1 to 30 carbon atoms, a substituted or unsubstituted alkenyl group having 2 to 30 carbon atoms, a substituted or unsubstituted cycloalkyl group having 3 to 30 carbon atoms, a substituted or unsubstituted cycloalkenyl group having 5 to 30 carbon atoms, a substituted or unsubstituted alkoxy group having 1 to 30 carbon atoms, a substituted or unsubstituted aryloxy group having 6 to 30 carbon atoms, a substituted or unsubstituted alkylthioxy group having 1 to 30 carbon atoms, a substituted or unsubstituted arylthioxy group having 6 to 30 carbon atoms, a substituted or unsubstituted alkylamine group having 1 to 30 carbon atoms, a substituted or unsubstituted arylamine group having 6 to 30 carbon atoms, a substituted or unsubstituted aryl group having 6 to 50 carbon atoms, a substituted or unsubstituted heteroaryl group having 3 to 50 carbon atoms, a substituted or unsubstituted alkylsilyl group having 1 to 30 carbon atoms, and a substituted or unsubstituted arylsilyl group having 6 to 30 carbon atoms,
n is 9, and
R1 to R8 are the same as or different from each other, and are each independently selected from the group consisting of a hydrogen atom, a deuterium atom, a substituted or unsubstituted alkyl group having 1 to 30 carbon atoms, a substituted or unsubstituted alkenyl group having 2 to 30 carbon atoms, a substituted or unsubstituted cycloalkyl group having 3 to 30 carbon atoms, a substituted or unsubstituted cycloalkenyl group having 5 to 30 carbon atoms, a substituted or unsubstituted aryl group having 6 to 50 carbon atoms, a substituted or unsubstituted heteroaryl group having 3 to 50 carbon atoms, a substituted or unsubstituted arylamine group having 6 to 30 carbon atoms, a substituted or unsubstituted alkylsilyl group having 1 to 30 carbon atoms, and a substituted or unsubstituted arylsilyl group having 6 to 30, and
wherein in Formula 1, “substituted” in “substituted or unsubstituted” means substituted with one or more substituents selected from the group consisting of a deuterium, a cyano group, a halogen group, a hydroxyl group, a nitro group, an alkyl group having 1 to 24 carbon atoms, an alkenyl group having 1 to 24 carbon atoms, a cycloalkyl group having 3 to 24 carbon atoms, a heteroalkyl group having 1 to 24 carbon atoms, an aryl group having 6 to 24 carbon atoms, an arylalkyl group having 7 to 24 carbon atoms, an alkylaryl group having 7 to 24 carbon atoms, a heteroaryl group having 2 to 24 carbon atoms, a heteroarylalkyl group having 2 to 24 carbon atoms, an alkoxy group having 1 to 24 carbon atoms, an alkylamino group having 1 to 24 carbon atoms, a diarylamino group having 12 to 24 carbon atoms, a diheteroarylamino group having 2 to 24 carbon atoms, an aryl (heteroaryl) amino group having 7 to 24 carbon atoms, an alkylsilyl group having 1 to 24 carbon atoms, an arylsilyl group having 6 to 24 carbon atoms, an aryloxy group having 6 to 24 carbon atoms, and an arylthionyl group having 6 to 24 carbon atoms.

2. The organic electroluminescent device according to claim 1, wherein A is selected from the group consisting of a substituted or unsubstituted aryl group having 6 to 30 carbon atoms, and a substituted or unsubstituted heteroaryl group having 3 to 30 carbon atoms, wherein L1 and L2 are the same as or different from each other, and are each independently selected from the group consisting of a single bond and a substituted or unsubstituted arylene group having 6 to 30 carbon atoms,
wherein n1 and n2 are respectively 1,
wherein Rs are the same as or different from each other, and are each independently selected from the group consisting of a hydrogen atom, a deuterium atom, a substituted or unsubstituted alkyl group having 1 to 8 carbon atoms, a substituted or unsubstituted aryl group having 6 to 40 carbon atoms, and a substituted or unsubstituted heteroaryl group having 3 to 30 carbon atoms, and
wherein R1 to R8 are the same as or different from each other, and are each independently selected from the group consisting of a hydrogen atom, a deuterium atom, a substituted or unsubstituted alkyl group having 1 to 8 carbon atoms, a substituted or unsubstituted aryl group having 6 to 18 carbon atoms, and a substituted or unsubstituted heteroaryl group having 3 to 18 carbon atoms.

3. The organic electroluminescent device according to claim 1, wherein the solvent includes at least one of a chlorine-based solvent, an ether-based solvent, an aromatic solvent, an aliphatic solvent, a ketone-based solvent, an ester-based solvent, an alcohol-based solvent, an amide-based solvent, and a benzoate-based solvent.

4. The organic electroluminescent device according to claim 1, wherein the compound represented by Formula 1 has a solubility in the solvent of 0.1 wt % to 50 wt %.

5. The organic electroluminescent device according to claim 1, wherein the host is a compound having a molecular weight of 500 or more.

6. The organic electroluminescent device according to claim 1, wherein the host includes at least one alkyl group.

7. The organic electroluminescent device according to claim 1, wherein the host includes at least one deuterium.

8. The organic electroluminescent device according to claim 1, wherein the host is any one of the following compounds 1 to 102:

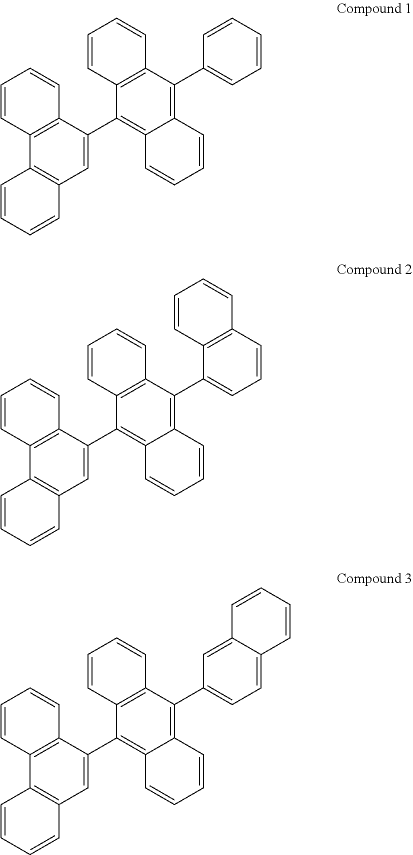

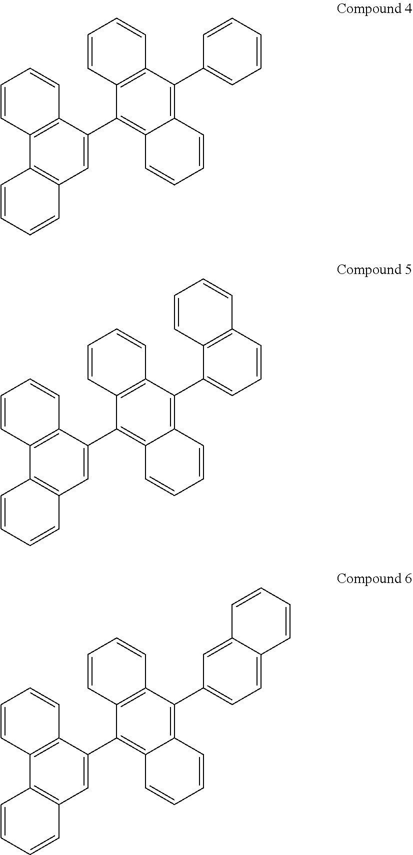

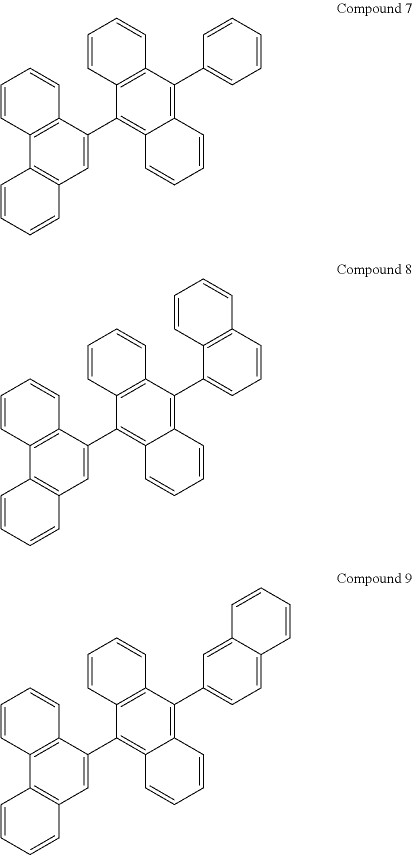

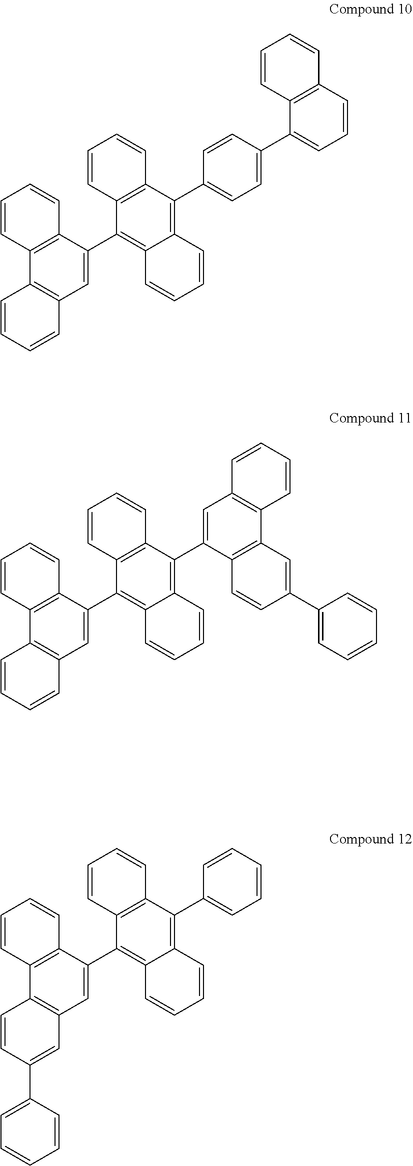

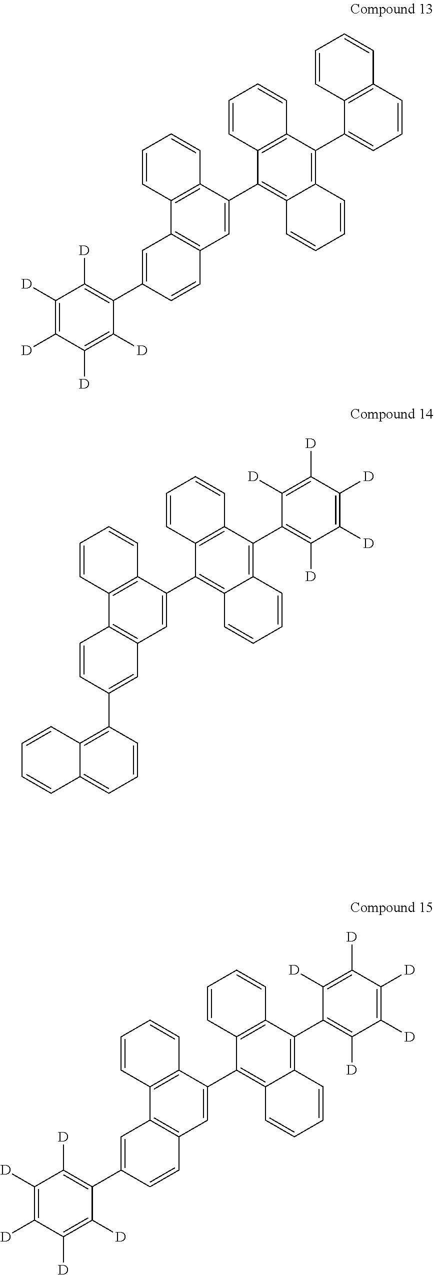 [SMILES: [2H]c1c([2H])c([2H])c(-c2ccc3cc(-c4c5ccccc5c(-c5cccc6ccccc56)c5ccccc45)c4ccccc4c3c2)c([2H])c1[2H] ; [2H]c1c([2H])c([2H])c(-c2c3ccccc3c(-c3cc4cc(-c5cccc6ccccc56)ccc4c4ccccc34)c3ccccc23)c([2H])c1[2H] ; [2H]c1c([2H])c([2H])c(-c2ccc3cc(-c4c5ccccc5c(-c5c([2H])c([2H])c([2H])c([2H])c5[2H])c5ccccc45)c4ccccc4c3c2)c([2H])c1[2H]]

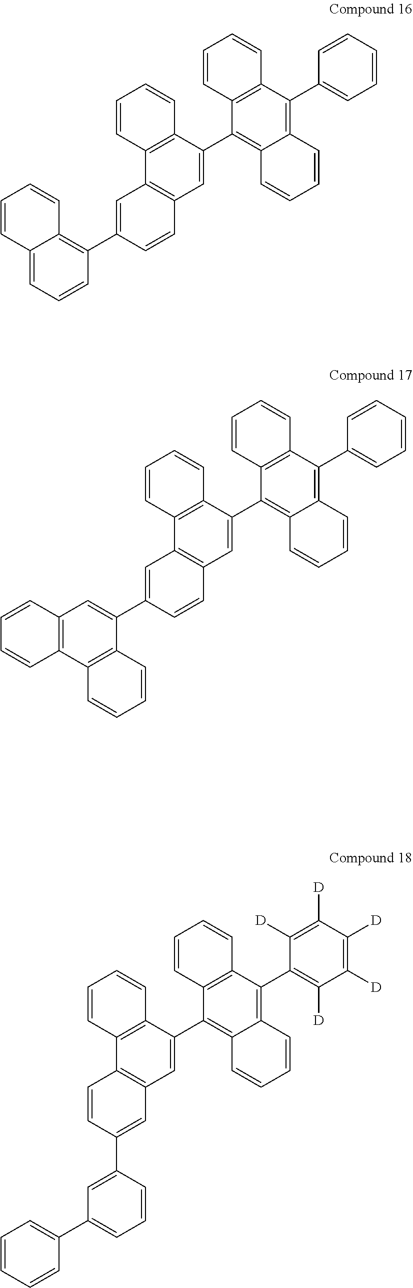

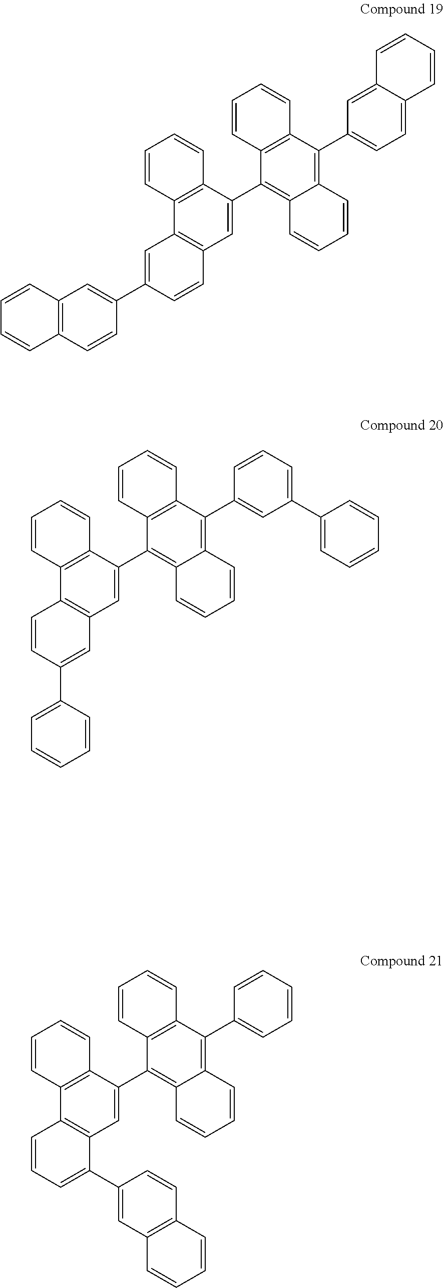

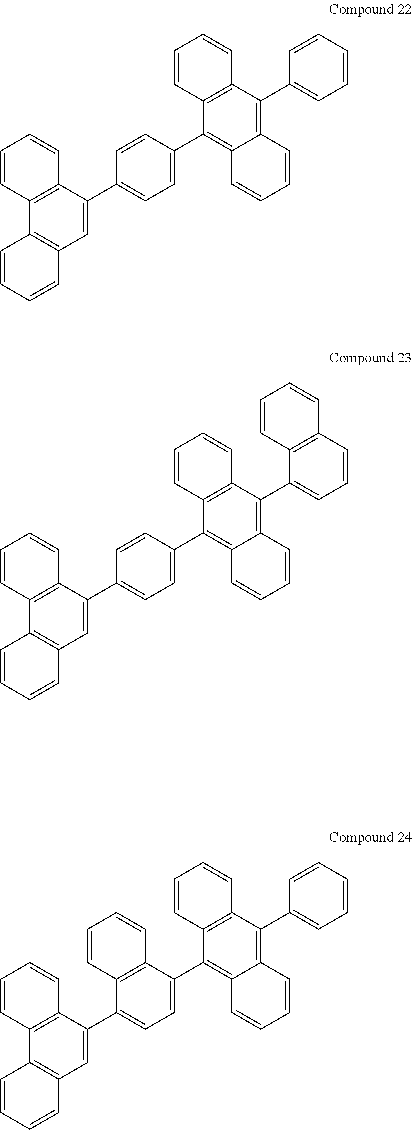

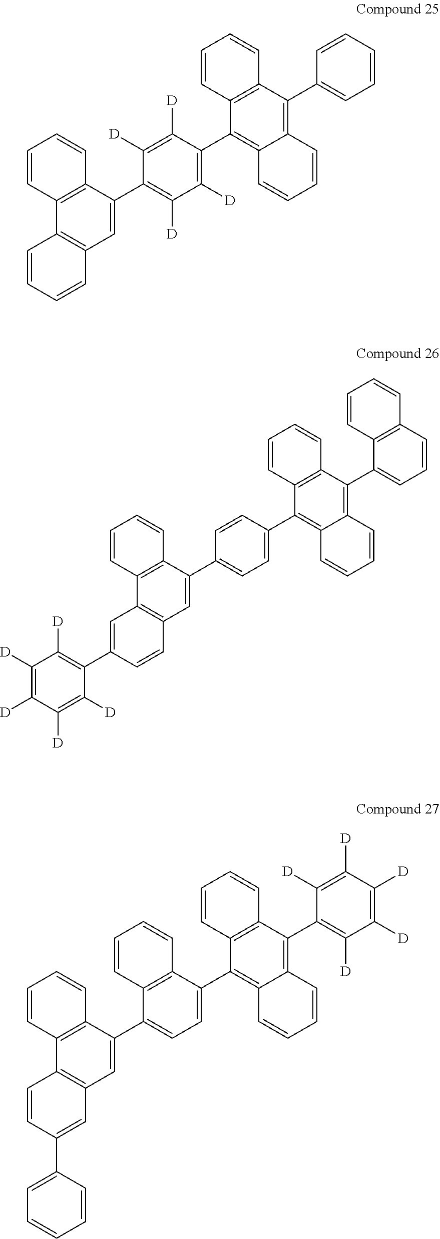 [SMILES: [2H]c1c([2H])c(-c2cc3ccccc3c3ccccc23)c([2H])c([2H])c1-c1c2ccccc2c(-c2ccccc2)c2ccccc12 ; [2H]c1c([2H])c([2H])c(-c2ccc3cc(-c4ccc(-c5c6ccccc6c(-c6cccc7ccccc67)c6ccccc56)cc4)c4ccccc4c3c2)c([2H])c1[2H] ; [2H]c1c([2H])c([2H])c(-c2c3ccccc3c(-c3ccc(-c4cc5cc(-c6ccccc6)ccc5c5ccccc45)c4ccccc34)c3ccccc23)c([2H])c1[2H]]

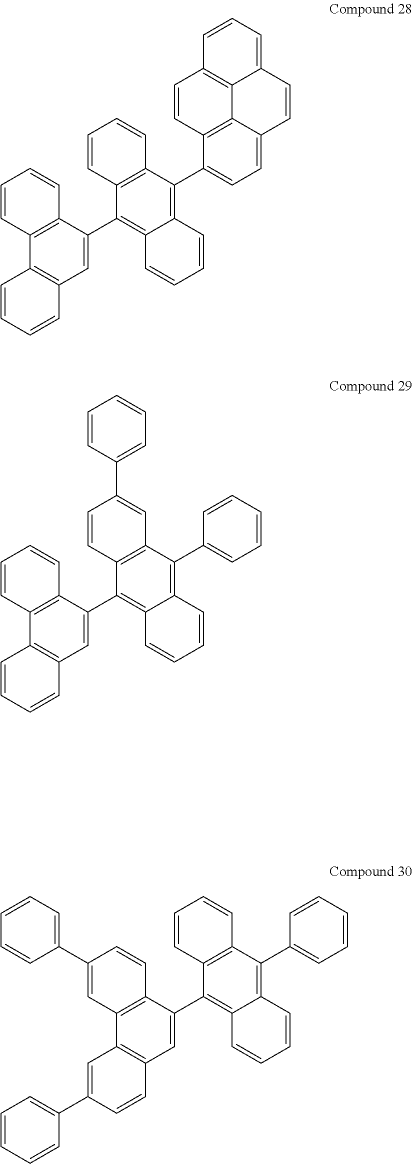

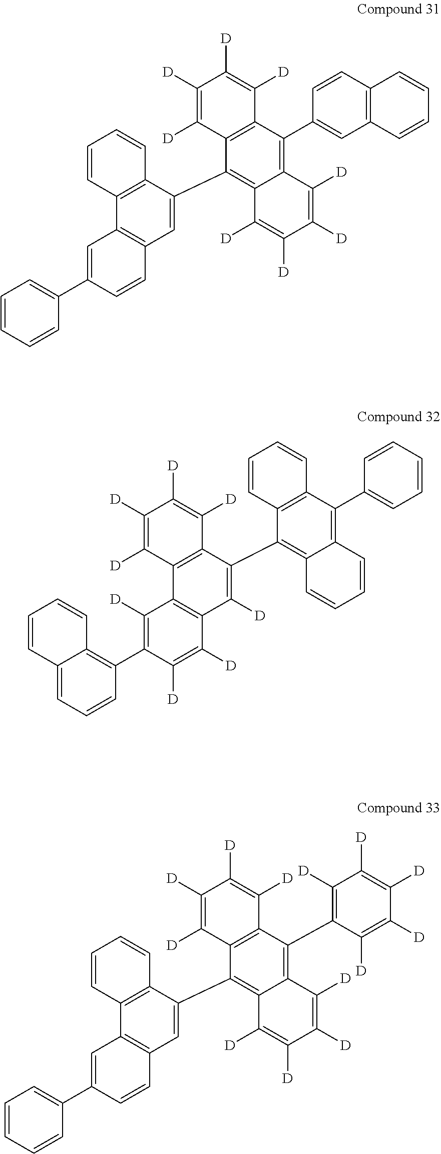 [SMILES: [2H]c1c([2H])c([2H])c2c(-c3cc4ccc(-c5ccccc5)cc4c4ccccc34)c3c([2H])c([2H])c([2H])c([2H])c3c(-c3ccc4ccccc4c3)c2c1[2H] ; [2H]c1c(-c2cccc3ccccc23)c([2H])c2c(c1[2H])c([2H])c(-c1c3ccccc3c(-c3ccccc3)c3ccccc13)c1c([2H])c([2H])c([2H])c([2H])c12 ; [2H]c1c([2H])c([2H])c(-c2c3c([2H])c([2H])c([2H])c([2H])c3c(-c3cc4ccc(-c5ccccc5)cc4c4ccccc34)c3c([2H])c([2H])c([2H])c([2H])c23)c([2H])c1[2H]]

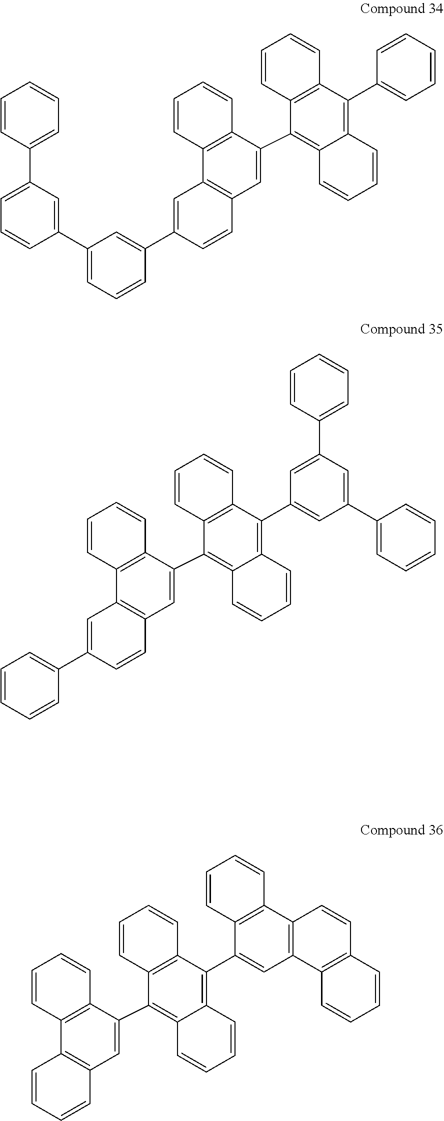

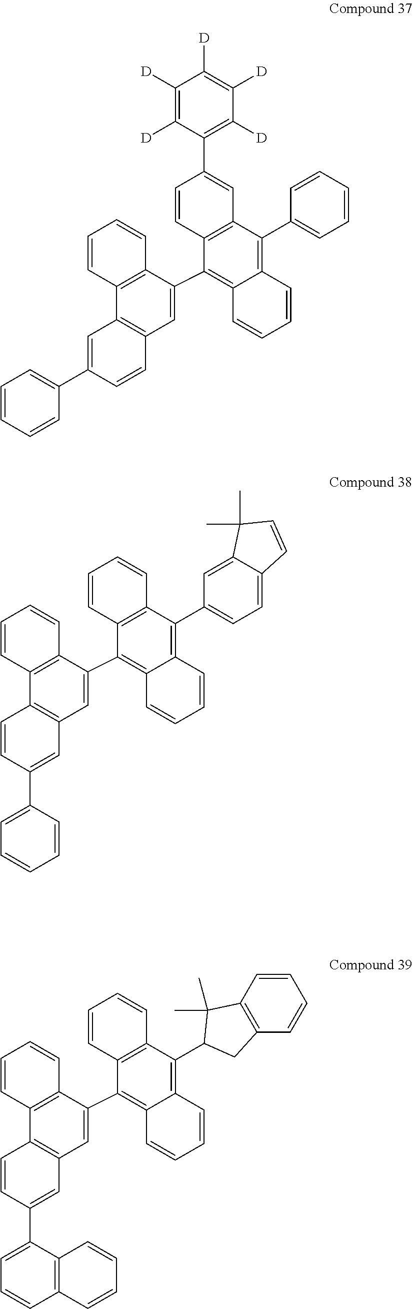

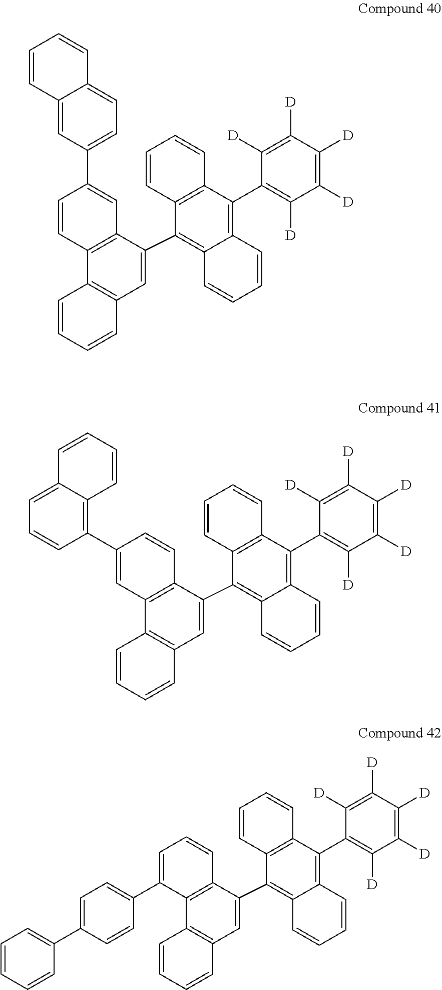 [SMILES: [2H]c1c([2H])c([2H])c(-c2c3ccccc3c(-c3cc4ccccc4c4ccc(-c5ccc6ccccc6c5)cc34)c3ccccc23)c([2H])c1[2H] ; [2H]c1c([2H])c([2H])c(-c2c3ccccc3c(-c3cc4ccccc4c4cc(-c5cccc6ccccc56)ccc34)c3ccccc23)c([2H])c1[2H] ; [2H]c1c([2H])c([2H])c(-c2c3ccccc3c(-c3cc4ccccc4c4c(-c5ccc(-c6ccccc6)cc5)cccc34)c3ccccc23)c([2H])c1[2H]]

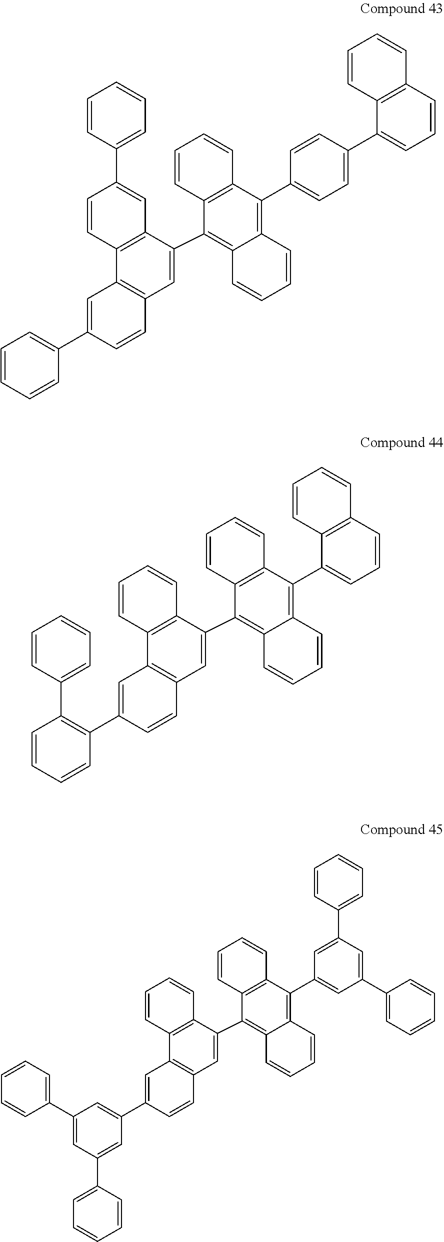

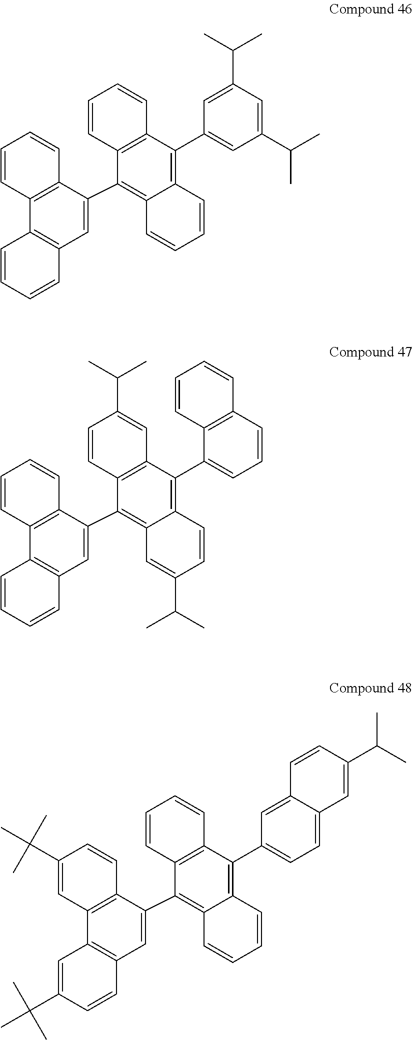

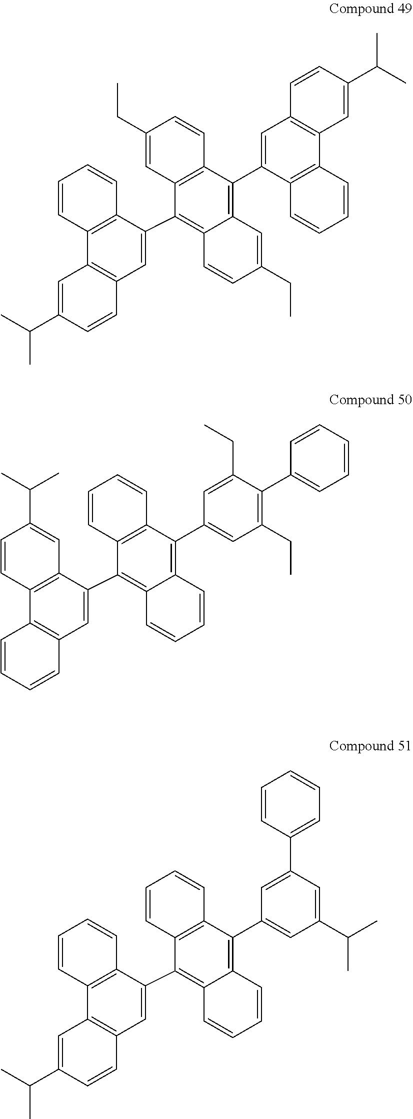

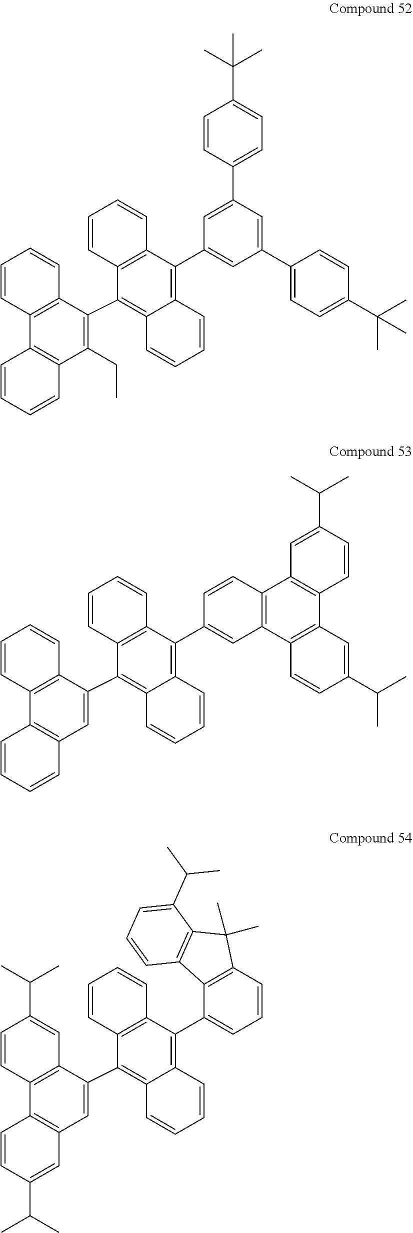

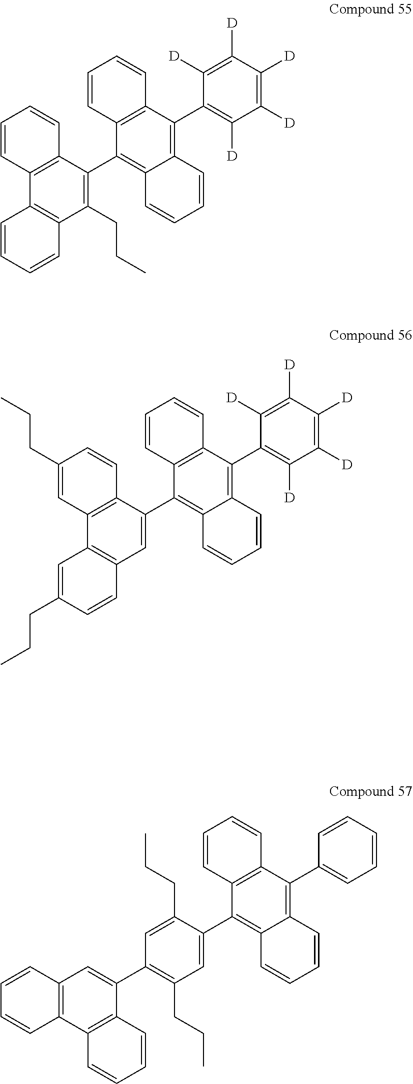

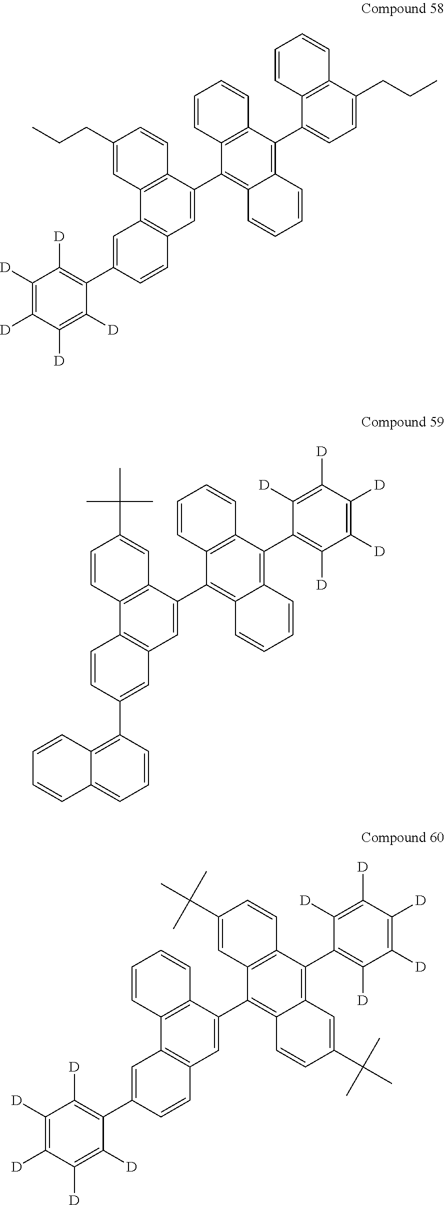 [SMILES: [2H]c1c([2H])c([2H])c(-c2ccc3cc(-c4c5ccccc5c(-c5ccc(CCC)c6ccccc56)c5ccccc45)c4ccc(CCC)cc4c3c2)c([2H])c1[2H] ; [2H]c1c([2H])c([2H])c(-c2c3ccccc3c(-c3cc4cc(-c5cccc6ccccc56)ccc4c4ccc(C(C)(C)C)cc34)c3ccccc23)c([2H])c1[2H] ; [2H]c1c([2H])c([2H])c(-c2ccc3cc(-c4c5ccc(C(C)(C)C)cc5c(-c5c([2H])c([2H])c([2H])c([2H])c5[2H])c5ccc(C(C)(C)C)cc45)c4ccccc4c3c2)c([2H])c1[2H]]

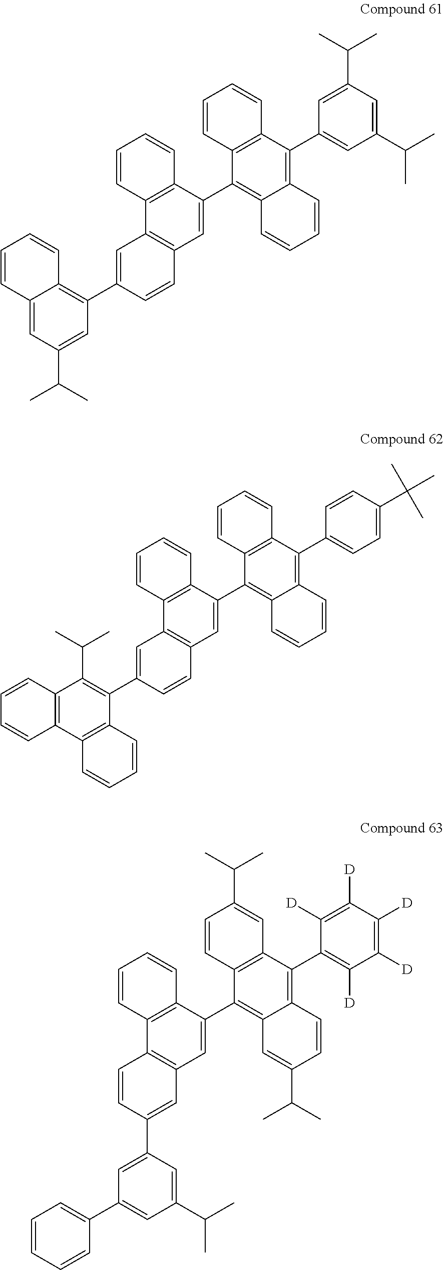

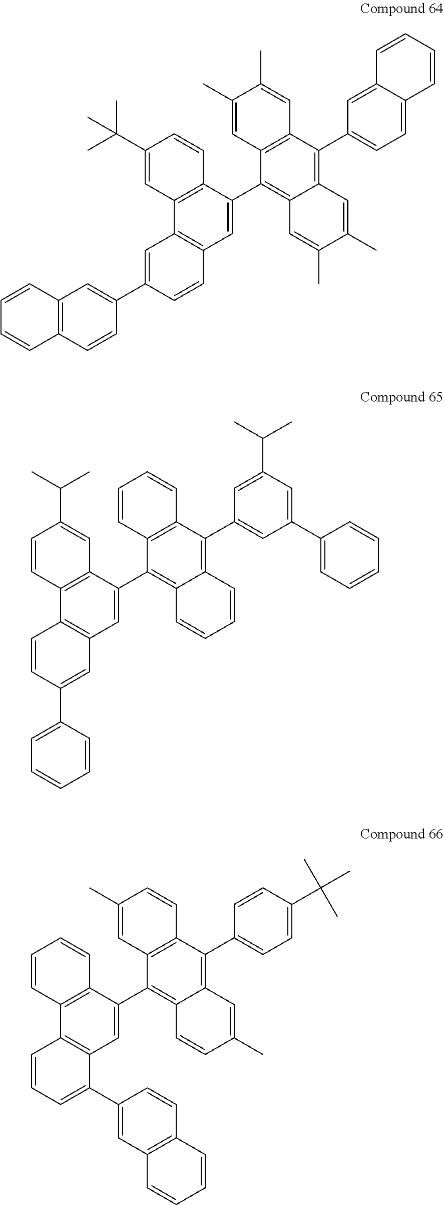

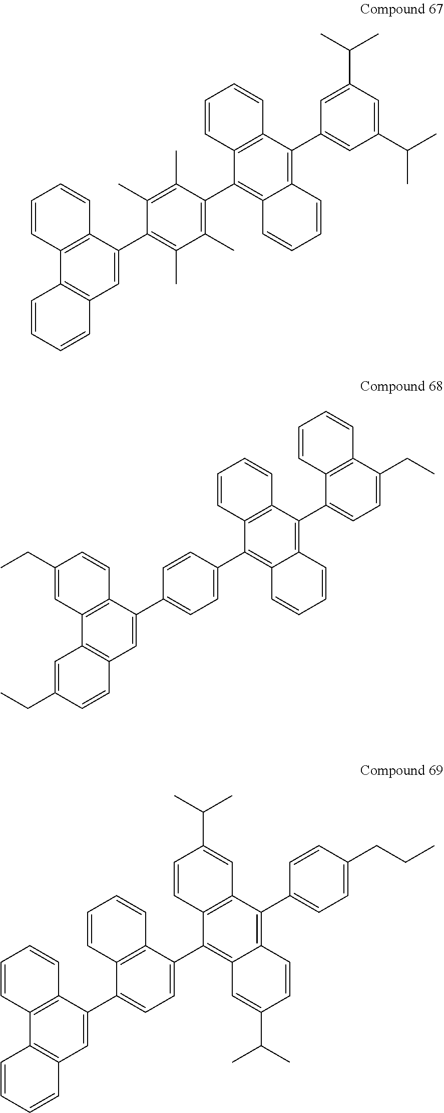

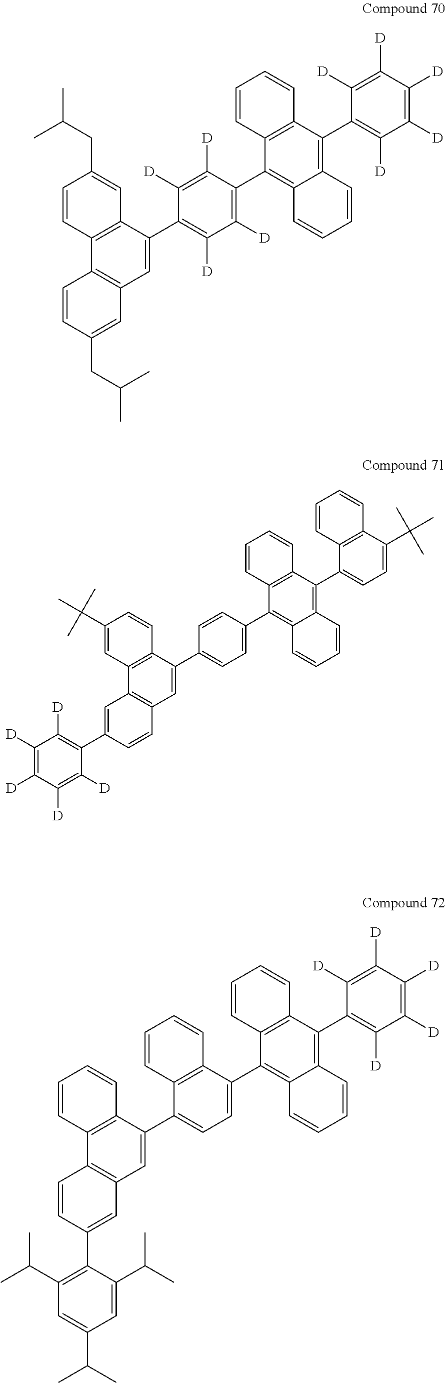

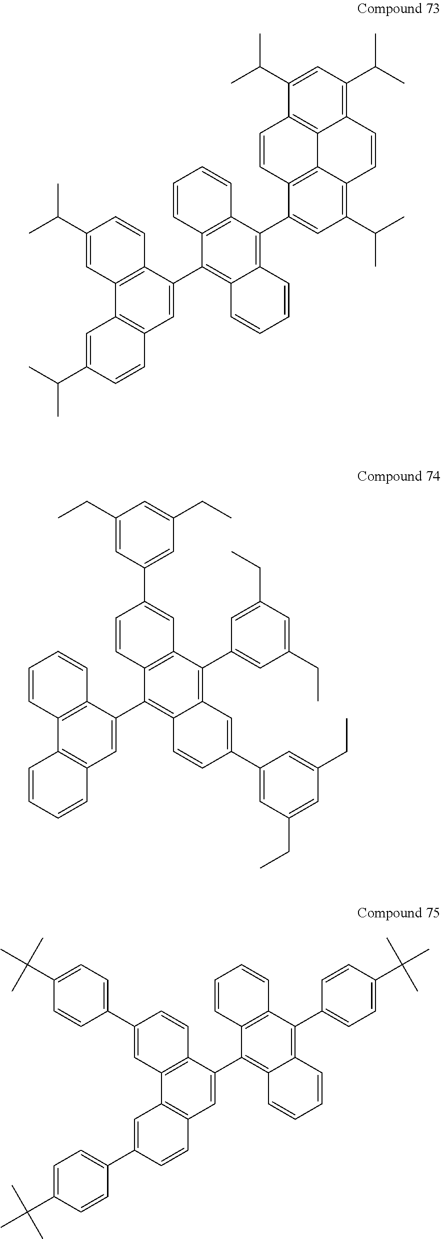

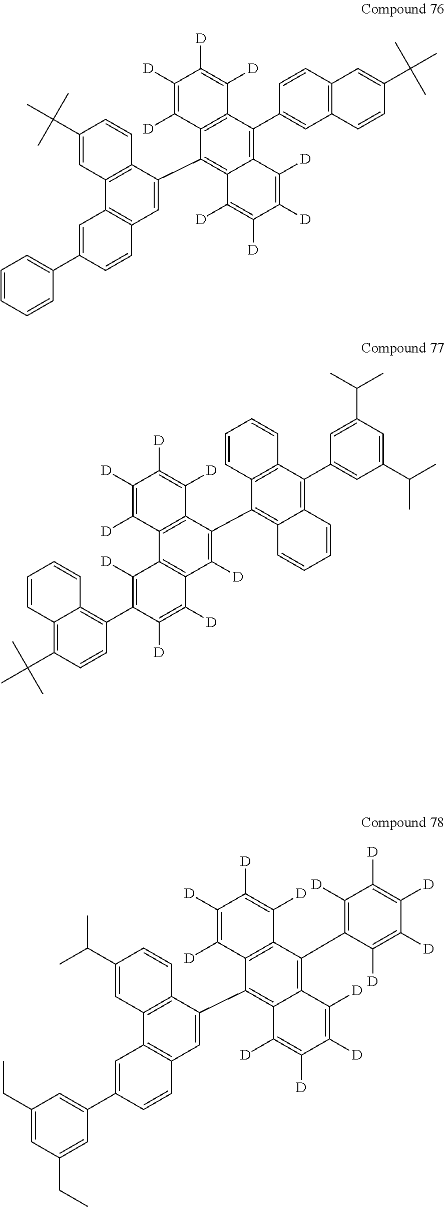

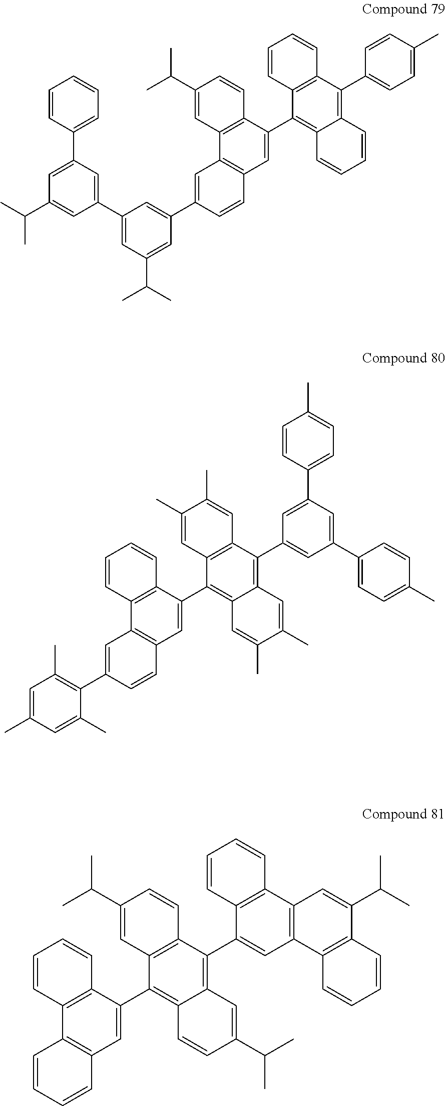

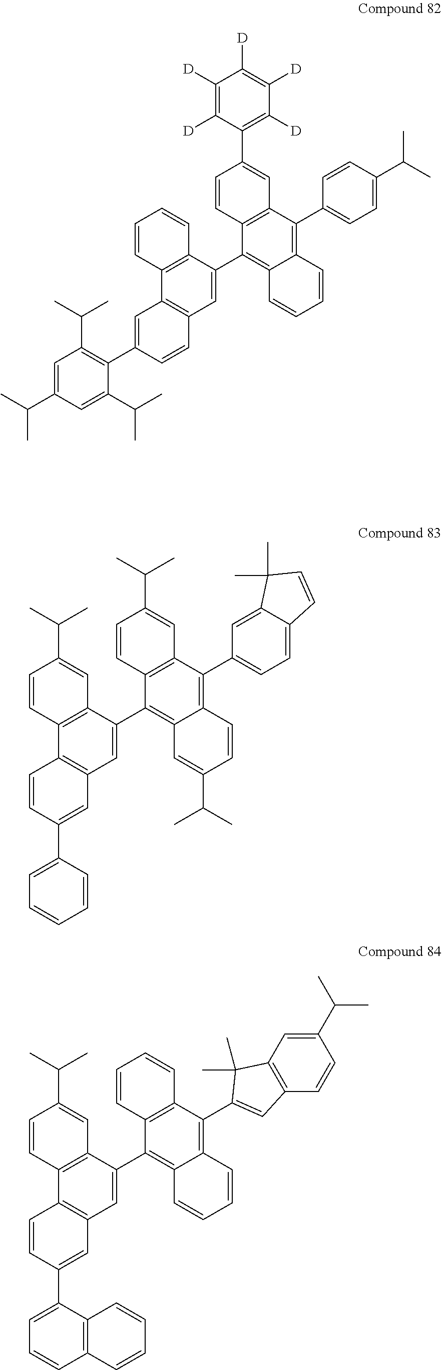

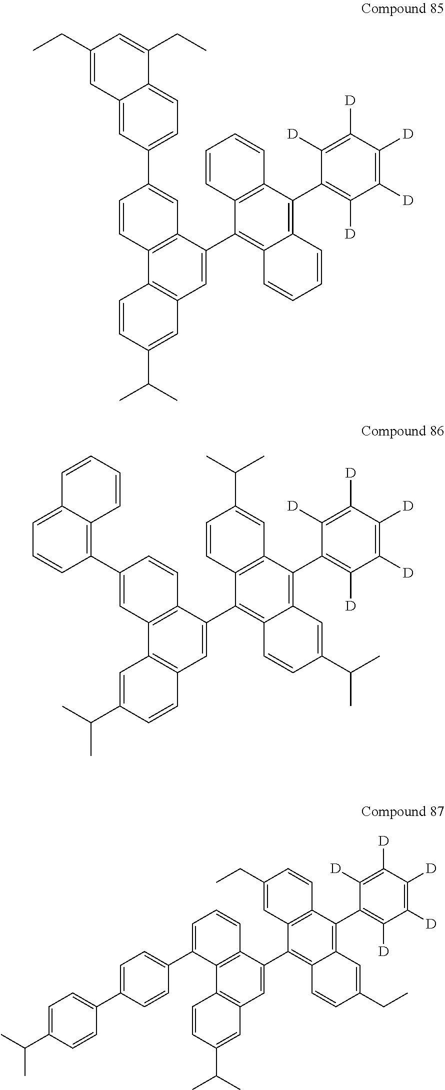 [SMILES: [2H]c1c([2H])c([2H])c(-c2c3ccccc3c(-c3cc4cc(C(C)C)ccc4c4ccc(-c5ccc6c(CC)cc(CC)cc6c5)cc34)c3ccccc23)c([2H])c1[2H] ; [2H]c1c([2H])c([2H])c(-c2c3cc(C(C)C)ccc3c(-c3cc4ccc(C(C)C)cc4c4cc(-c5cccc6ccccc56)ccc34)c3ccc(C(C)C)cc23)c([2H])c1[2H] ; [2H]c1c([2H])c([2H])c(-c2c3cc(CC)ccc3c(-c3cc4cc(C(C)C)ccc4c4c(-c5ccc(-c6ccc(C(C)C)cc6)cc5)cccc34)c3cc(CC)ccc23)c([2H])c1[2H]]

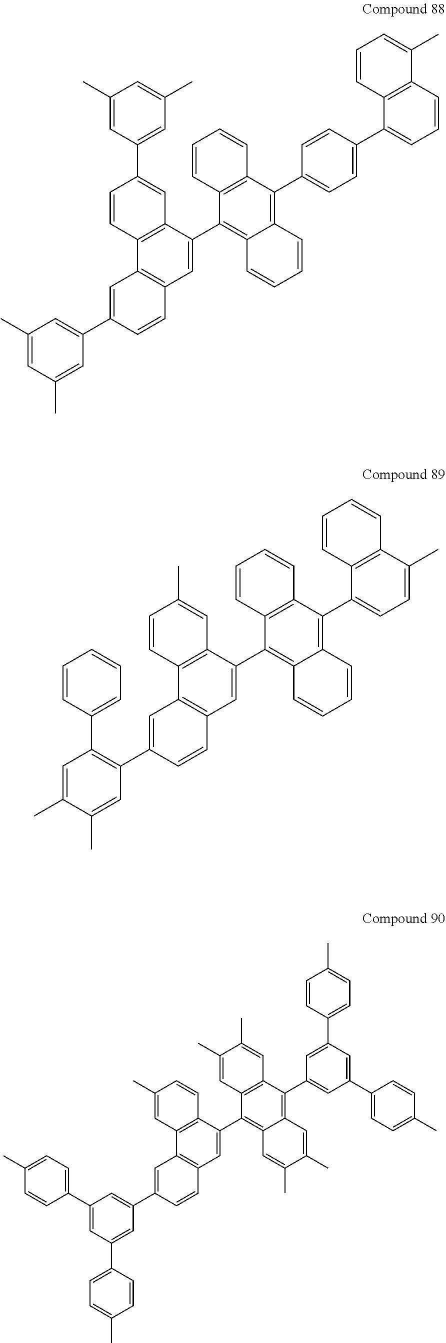

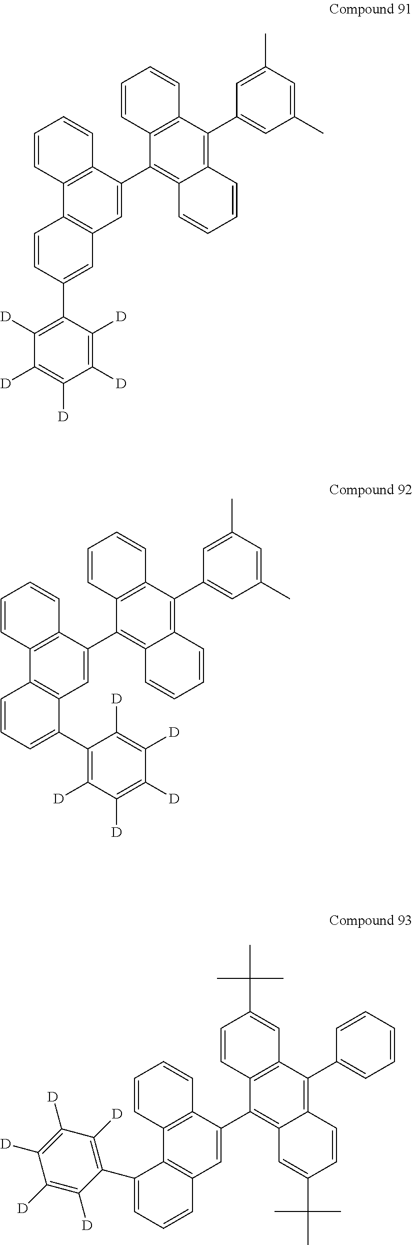 [SMILES: [2H]c1c([2H])c([2H])c(-c2ccc3c(c2)cc(-c2c4ccccc4c(-c4cc(C)cc(C)c4)c4ccccc24)c2ccccc23)c([2H])c1[2H] ; [2H]c1c([2H])c([2H])c(-c2cccc3c2cc(-c2c4ccccc4c(-c4cc(C)cc(C)c4)c4ccccc24)c2ccccc23)c([2H])c1[2H] ; [2H]c1c([2H])c([2H])c(-c2cccc3cc(-c4c5ccc(C(C)(C)C)cc5c(-c5ccccc5)c5ccc(C(C)(C)C)cc45)c4ccccc4c23)c([2H])c1[2H]]

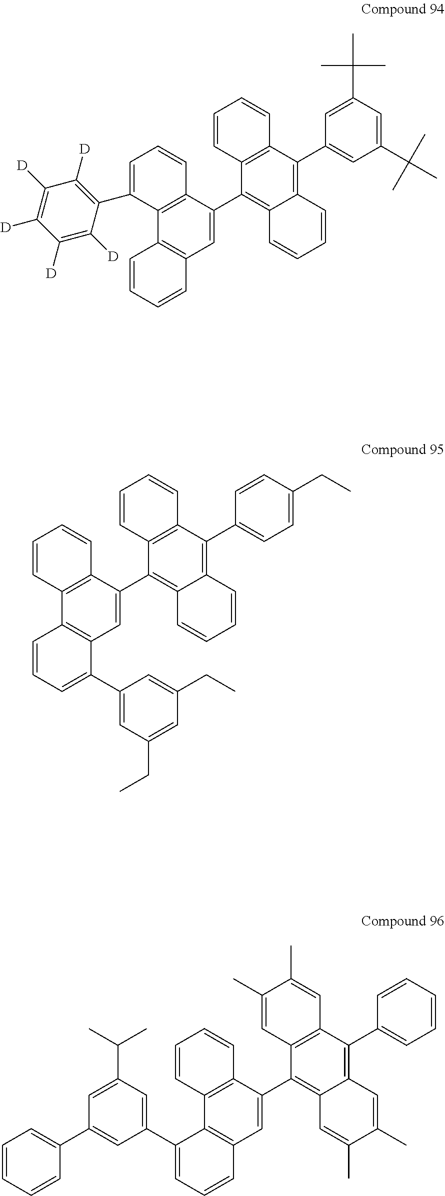

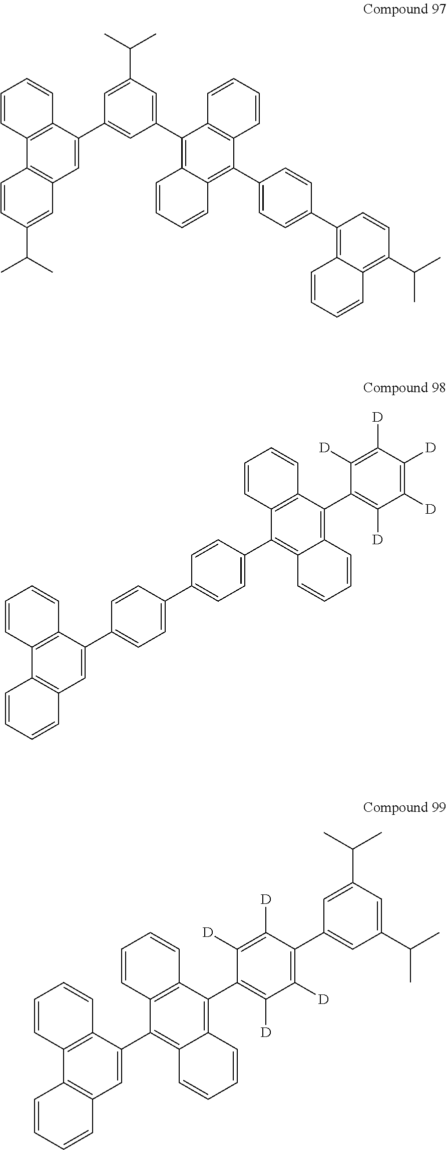

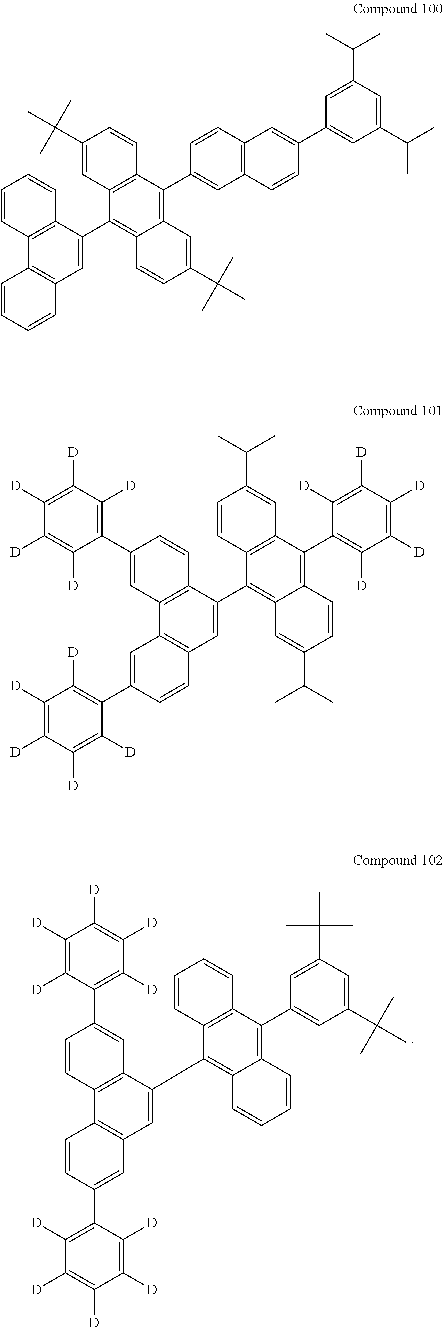

9. The organic electroluminescent device according to claim 1, wherein the composition further comprises at least one dopant.

10. The organic electroluminescent device according to claim 1, wherein the organic material layer is formed by any one of spin coating, dip coating, doctor blade coating, spray coating, roll coating, inkjet printing and screen printing.
